(19) United States
(12) Patent Application Publication
(10) Pub. No.: US 2024/0155241 A1
(43) Pub. Date: May 9, 2024

(54) MEDICAL OBSERVATION SYSTEM, INFORMATION PROCESSING DEVICE, AND INFORMATION PROCESSING METHOD

(71) Applicant: SONY GROUP CORPORATION, TOKYO (JP)

(72) Inventor: MASARU USUI, TOKYO (JP)

(21) Appl. No.: 18/568,862

(22) PCT Filed: Feb. 14, 2022
(86) PCT No.: PCT/JP2022/005677

§ 371 Date: Dec. 11, 2023

(30) Foreign Application Priority Data
Jun. 21, 2021 (JP) 2021-102329

(51) Int. Cl. H04N 23/695; A61B 1/00; G06T 7/73

(52) U.S. Cl. CPC H04N 23/695; A61B 1/000094; A61B 1/00149; G06T 7/73; G06T 2207/10068; G06T 2207/30244

(57) ABSTRACT

A medical observation system according to an aspect of the present disclosure includes: an endoscope that acquires a first operative field image; an arm unit that supports and moves the endoscope; a gaze target extraction unit (271a) that extracts a gaze target from the first operative field image; a gaze point information calculation unit (271b) that calculates gaze point information related to a gaze point of the gaze target; a movable range determination unit (272a) that determines, on the basis of the gaze point information, a movable range of the endoscope enabling cutout of a second operative field image including the gaze point from the first operative field image; a camera posture determination unit (272b) being an example of a posture determination unit that determines posture information related to a position and a posture of the endoscope on the basis of the movable range; and an arm control unit (23) that controls the arm unit on the basis of the posture information.

### FIELD

The present disclosure relates to a medical observation system, an information processing device, and an information processing method.

### BACKGROUND

In recent years, in endoscopic surgery, the abdominal cavity of a patient is imaged by an endoscope such as a fixed oblique-viewing angle endoscope or a variable oblique-viewing angle endoscope, and the captured image of the abdominal cavity is displayed by a display. The surgeon performs surgery while monitoring the captured image displayed on the display. For example, Patent Literature 1 below discloses a technique of appropriately controlling an arm that supports an endoscope on the basis of a captured image.

### CITATION LIST

### Patent Literature

Patent Literature 1: JP 2021-13412 A

### SUMMARY

### Technical Problem

Normally, the fixed oblique-viewing angle endoscope cannot capture a gaze target at the center in a multiple viewpoint under the trocar constraint condition. In addition, in order to simultaneously capture multiple gaze targets, the endoscope operation range is limited, making it difficult to continuously operate the endoscope while continuously capturing all the gaze targets in the screen. In addition, while a multiple viewpoint is enabled with the variable oblique-viewing angle endoscope, multiple gaze targets cannot be captured from a requested gaze line direction. This makes it difficult to determine an optimum endoscope position/posture for continuously capturing the gaze target in the camera or determine an oblique-viewing angle that defines a gaze line vector. These situations make it difficult to capture the gaze target in the visual field in an appropriate gaze line direction.

In view of this, the present disclosure proposes a medical observation system, an information processing device, and an information processing method capable of capturing a gaze target in a visual field in an appropriate gaze line direction.

### Solution to Problem

A medical observation system according to the embodiment of the present disclosure includes: an endoscope that acquires a first operative field image; an arm unit that supports and moves the endoscope; a gaze target extraction unit that extracts a gaze target from the first operative field image; a gaze point information calculation unit that calculates gaze point information related to a gaze point of the gaze target; a movable range determination unit that determines, on the basis of the gaze point information, a movable range of the endoscope enabling cutout of a second operative field image including the gaze point from the first operative field image; a posture determination unit that determines posture information related to a position and a posture of the endoscope on the basis of the movable range; and an arm control unit that controls the arm unit on the basis of the posture information.

An information processing device according to the embodiment of the present disclosure includes: a gaze target extraction unit that extracts a gaze target from a first operative field image obtained by an endoscope; a gaze point information calculation unit that calculates gaze point information related to a gaze point of the gaze target; a movable range determination unit that determines, on the basis of the gaze point information, a movable range of the endoscope enabling cutout of a second operative field image including the gaze point from the first operative field image; a posture determination unit that determines posture information related to a position and a posture of the endoscope on the basis of the movable range; and an arm control unit that controls an arm unit that supports and moves the endoscope, on the basis of the posture information.

An information processing method according to the embodiment of the present disclosure includes: extracting a gaze target from a first operative field image obtained by an endoscope; calculating gaze point information related to a gaze point of the gaze target; determining, on the basis of the gaze point information, a movable range of the endoscope enabling cutout of a second operative field image including the gaze point from the first operative field image; determining posture information related to a position and a posture of the endoscope on the basis of the movable range; and controlling an arm unit that supports and moves the endoscope, on the basis of the posture information.

### BRIEF DESCRIPTION OF DRAWINGS

FIG. 1 is a diagram illustrating an example of a schematic configuration of an endoscopic surgery system according to an embodiment of the present disclosure.

FIG. 2 is a diagram illustrating an example of a detailed configuration of a camera head and a CCU according to the embodiment of the present disclosure.

FIG. 3 is a diagram illustrating an example of an external configuration of the support arm device according to the embodiment of the present disclosure.

FIG. 4 is a diagram illustrating an example of a schematic configuration of a medical observation system according to the embodiment of the present disclosure.

FIG. 5 is a diagram illustrating an example of a detailed configuration of a robot arm device according to the embodiment of the present disclosure.

FIG. 6 is a diagram illustrating an example of a flow of processing of the medical observation system according to the embodiment of the present disclosure.

FIG. 7 is a diagram illustrating an example of generating a wide-angle image and a cutout image according to the embodiment of the present disclosure.

FIG. 8 is a diagram illustrating an example of a detailed configuration of a gaze processing unit according to the embodiment of the present disclosure.

FIG. 9 is a flowchart illustrating an example of basic processing according to the embodiment of the present disclosure.

FIG. 10 is a diagram illustrating gaze point information calculation according to the embodiment of the present disclosure.

FIG. 11 is a diagram illustrating an example in which a feature point is missing due to an obstacle according to the embodiment of the present disclosure.

FIG. 12 is a diagram illustrating a cutout maximum oblique-viewing angle according to the embodiment of the present disclosure.

FIG. 13 is a diagram illustrating endoscope movable range determination of a single gaze point according to the embodiment of the present disclosure.

FIG. 14 is a diagram illustrating determination of an endoscope movable range of multiple gaze points according to the embodiment of the present disclosure.

FIG. 15 is a diagram illustrating an endoscope distal end requested movement trajectory in a case where a requested gaze line vector is within an endoscope movable range according to the embodiment of the present disclosure.

FIG. 16 is a diagram illustrating an endoscope distal end requested movement trajectory in a case where a requested gaze line vector is outside an endoscope movable range according to the embodiment of the present disclosure.

FIG. 17 is a diagram illustrating an endoscope distal end requested movement trajectory in a case where a requested gaze line vector of each gaze point is within an endoscope movable range according to the embodiment of the present disclosure.

FIG. 18 is a diagram illustrating an endoscope distal end requested movement trajectory in a case where a requested gaze line vector of each gaze point is outside an endoscope movable range according to the embodiment of the present disclosure.

FIG. 19 is a flowchart illustrating a flow of processing of calculating an average requested gaze line vector of all gaze points and performing tracking according to the embodiment of the present disclosure.

FIG. 20 is a diagram illustrating an endoscope distal end position and a cutout gaze line vector at the time of multiple gaze point cutout according to the embodiment of the present disclosure.

FIG. 21 is a diagram illustrating an example of an image at the time of multiple gaze point cutout according to the embodiment of the present disclosure.

FIG. 22 is a diagram illustrating generation of a straight-viewing cutout gaze line vector of a single gaze point according to a first modification of the embodiment of the present disclosure.

FIG. 23 is a diagram illustrating distal end position determination according to a requested level (ratio) of multiple gaze points according to the first modification of the embodiment of the present disclosure.

FIG. 24 is a flowchart illustrating a flow of processing of a requested gaze line vector non-reference case according to the first modification of the embodiment of the present disclosure.

FIG. 25 is a diagram illustrating virtual wall setting by an endoscope movable range of multiple gaze points according to a second modification of the embodiment of the present disclosure.

FIG. 26 is a diagram illustrating a contact avoidance operation by endoscope approach prohibition distance setting according to the second modification of the embodiment of the present disclosure.

FIG. 27 is a flowchart illustrating a flow of processing of a virtual wall setting case based on endoscope movable range information according to the second modification of the embodiment of the present disclosure.

FIG. 28 is a diagram illustrating minimization of an endoscope posture change amount at the time of cutout visual field movement according to a third modification of the embodiment of the present disclosure.

FIG. 29 is a diagram illustrating an example of a schematic configuration of hardware.

### DESCRIPTION OF EMBODIMENTS

Embodiments of the present disclosure will be described below in detail with reference to the drawings. Note that the system, the device, the method, and the like according to the present disclosure are not limited by the embodiment. In addition, basically, redundant descriptions will be omitted from the present specification and the drawings by assigning the same reference signs to components having substantially the same functional configuration.

One or more embodiments (including examples and modifications) described below can each be implemented independently. On the other hand, at least some of the multiple embodiments described below may be appropriately combined with at least some of other embodiments. The multiple embodiments may include novel features different from each other. Accordingly, the multiple embodiments can contribute to achieving or solving different objects or problems, and can exhibit different effects.

The present disclosure will be described in the following order.
1. Embodiments
1-1. Configuration example of endoscopic surgery system
1-1-1. Schematic configuration example of endoscopic surgery system
1-1-2. Detailed configuration example of support arm device
1-1-3. Detailed configuration example of light source device
1-1-4. Detailed configuration example of camera head and
CCU
1-1-5. Example of external configuration of support arm device
1-2. Configuration of medical observation system
1-2-1. Schematic configuration example of medical observation system
1-2-2. Detailed configuration example of robot arm device
1-2-3. Processing example of medical observation system
1-2-4. Example of generation processing of wide-angle image and cutout image
1-2-5. Detailed configuration example of gaze processing unit
1-2-6. Detailed processing example of gaze processing unit
1-3. First modification
1-4. Second modification
1-5. Third modification
1-6. Action and effect
2. Other embodiments
3. Hardware configuration example
4. Supplementary notes

### 1. EMBODIMENTS

<1-1. Configuration Example of Endoscopic Surgery System>

<1-1-1. Schematic Configuration Example of Endoscopic Surgery System>

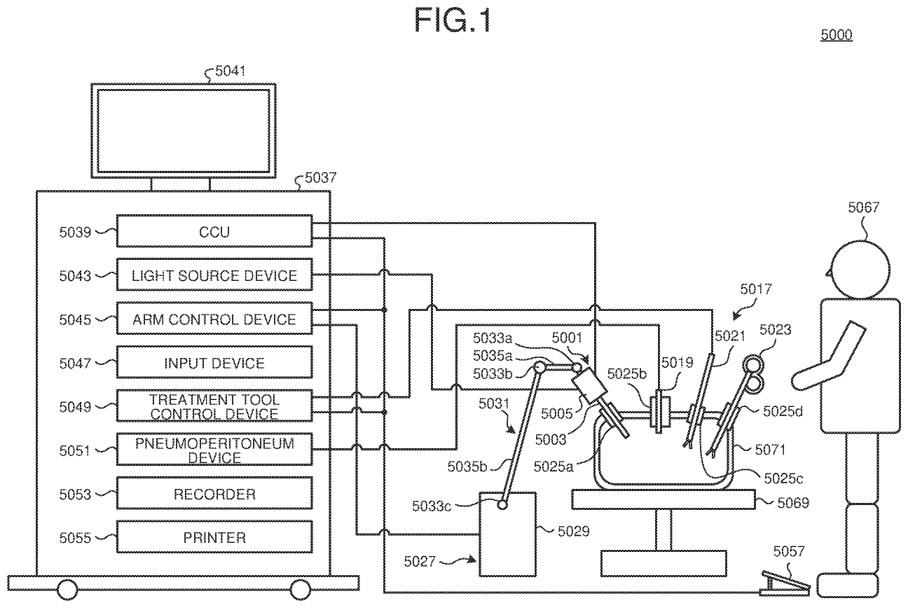

An example of a schematic configuration of an endoscopic surgery system 5000 according to the present embodiment will be described with reference to FIG. 1. FIG. 1 is a diagram illustrating an example of a schematic configuration of the endoscopic surgery system 5000 according to the present embodiment.

FIG. 1 illustrates a scene in which a surgeon (doctor) 5067 is performing surgery on a patient 5071 on a patient bed 5069 using the endoscopic surgery system 5000. As illustrated in FIG. 1, the endoscopic surgery system 5000 includes: an endoscope 5001, other surgical tools 5017; a support arm device 5027 that supports the endoscope 5001; and a cart 5037 equipped with various devices for endoscopic surgery.

In endoscopic surgery, an abdominal wall is punctured with multiple tubular laparotomy instruments referred to as trocars 5025a to 5025d, for example, instead of a method of cutting the abdominal wall for open surgery. Through the trocars 5025a to 5025d, a lens barrel 5003 of the endoscope 5001 and other surgical tools 5017 are inserted into the body cavity of the patient 5071. In the example of FIG. 1, as other surgical tools 5017, an insufflation tube 5019, an energy treatment tool 5021 and forceps 5023 are being inserted into the body cavity of the patient 5071. Furthermore, the energy treatment tool 5021 is a treatment tool used for incision and detachment of tissues, blood vessel sealing, or the like, by using high-frequency current or ultrasonic vibration. Note that the surgical tool 5017 illustrated in FIG. 1 is just an example, and other applicable examples of the surgical tool 5017 include various surgical tools generally used in endoscopic surgery, such as tweezers and a retractor.

An image of the surgical site in the body cavity of the patient 5071 captured by the endoscope 5001 is displayed on a display device 5041. While viewing the surgical site image displayed on the display device 5041 in real time, the surgeon 5067 performs procedures such as resecting the affected part by using the energy treatment tool 5021 and the forceps 5023. Although not illustrated, the insufflation tube 5019, the energy treatment tool 5021, and the forceps 5023 are supported by a person such as the surgeon 5067 and assistants, for example, during the surgery.

### (Support Arm Device)

The support arm device 5027 includes an arm unit 5031 extending from a base unit 5029. In the example of FIG. 1, the arm unit 5031 includes joints 5033a, 5033b, and 5033c and links 5035a and 5035b, and is driven under the control of an arm control device 5045. The arm unit 5031 supports the endoscope 5001 and controls its position and posture. This makes it possible to stabilize the position of the endoscope 5001.

### (Endoscope)

The endoscope 5001 includes a lens barrel 5003 having a region of a predetermined length from a distal end thereof to be inserted into a body cavity of the patient 5071, and a camera head 5005 connected to a proximal end of the lens barrel 5003. The example of FIG. 1 illustrates the endoscope 5001 as a rigid endoscope having the lens barrel 5003 of a rigid type. However, the endoscope 5001 is not particularly limited and can be a flexible endoscope having the lens barrel 5003 of a flexible material.

The distal end of the lens barrel 5003 has an aperture to which an objective lens is fitted. A light source device 5043 is connected to the endoscope 5001 such that light generated by the light source device 5043 is introduced to a distal end of the lens barrel 5003 by a light guide extending in the inside of the lens barrel 5003 and is emitted toward an observation target in a body cavity of the patient 5071 through the objective lens. Note that the endoscope 5001 may be a straight-viewing endoscope, an oblique-viewing endoscope, or a side-viewing endoscope, and is not particularly limited.

An optical system and an imaging element (for example, image sensor) are provided in the inside of the camera head 5005 such that reflected light (observation light) from the observation target is condensed on the imaging element by the optical system. The observation light is photo-electrically converted by the imaging element to generate an electric signal corresponding to the observation light, namely, an image signal corresponding to an observation image. The image signal is transmitted as RAW data to a camera control unit (CCU) 5039. The camera head 5005 has a function of adjusting a magnification and a focal length by appropriately driving the optical system.

Incidentally, the camera head 5005 may include multiple imaging elements in order to support stereoscopic viewing (3D display) or the like. In this case, multiple relay optical systems is provided inside the lens barrel 5003 in order to guide the observation light to each of the multiple imaging elements.

### (Various Devices Mounted on Cart)

The CCU 5039 includes a central processing unit (CPU), a graphics processing unit (GPU) or the like and comprehensively controls operation of the endoscope 5001 and a display device 5041. Specifically, the CCU 5039 applies, on the image signal received from the camera head 5005, various types of image processing for displaying an image based on the image signal, such as developing processing (demosaicing). The CCU 5039 provides the image signal that has undergone the image processing to the display device 5041. Furthermore, the CCU 5039 transmits a control signal to the camera head 5005 and controls driving thereof. The control signal can include information regarding imaging conditions such as magnification and focal length.

Under the control of the CCU 5039, the display device 5041 displays an image based on the image signal that has undergone the image processing performed by the CCU 5039. When the endoscope 5001 is a device compatible with high-resolution imaging such as 4K (the number of horizontal pixels 3840×the number of vertical pixels 2160) or 8K (the number of horizontal pixels 7680×the number of vertical pixels 4320), and/or when the endoscope 5001 is a device compatible with 3D display, for example, the display device 5041 can be a display device capable of high-resolution display and/or capable of 3D display, corresponding to individual specs. When the endoscope 5001 is a device compatible with high resolution imaging such as 4K or 8K, using the display device 5041 having a size of 55 inches or more can obtain further immersive feeling. Furthermore, the display device 5041 may be provided in plurality, each having different resolutions and sizes for different applications.

The light source device 5043 includes a light source such as, for example, a light emitting diode (LED) and supplies irradiation light upon imaging of a surgical site to the endoscope 5001.

The arm control device 5045 includes, for example, a processor such as a CPU, and operates according to a predetermined program to control drive of the arm unit 5031 of the support arm device 5027 according to a predetermined control method.

An input device 5047 is an input interface for the endoscopic surgery system 5000. The user can input various types of information and input instructions to the endoscopic surgery system 5000 via the input device 5047. For example, the user inputs various types of information related to the surgery, such as physical information regarding the patient and information regarding the surgical procedure, via the input device 5047. Furthermore, the user inputs, through the input device 5047, an instruction to drive the arm unit 5031, an instruction to change imaging conditions (type of irradiation light, magnification, focal length, or the like) of the endoscope 5001, and an instruction to drive the energy treatment tool 5021, for example.

The type of the input device 5047 is not limited, and the input device 5047 may be various known input devices. Examples of applicable input devices 5047 include a mouse, a keyboard, a touch panel, a switch, a foot switch 5057, and/or a lever. When a touch panel is used as the input device 5047, the touch panel may be provided on a display surface of the display device 5041. Alternatively, the input device 5047 is a device worn by the user (the surgeon 5067, for example), such as an eyeglass type wearable device or head mounted display (HMD), for example. Various types of inputs are performed in accordance with user's gesture and gaze line detected by these devices. The input device 5047 includes a camera capable of detecting the movement of the user. Various types of inputs are performed in accordance with the user's gesture and gaze line detected from a video image captured by the camera. Furthermore, the input device 5047 includes a microphone capable of capturing user's voice, and various inputs are performed by voice through the microphone. In this manner, with a configuration of the input device 5047 capable of inputting various types of information in a non-contact manner, it is possible for the user (for example, the surgeon 5067) located in a clean area to perform non-contact operation of a device located in an unclean area. In addition, since the user can operate the device without releasing a hand from one's surgical tool, leading to enhancement of convenience for the user.

A treatment tool control device 5049 controls the drive of the energy treatment tool 5021 for ablation or dissection of tissue, sealing of blood vessels, and the like. A pneumoperitoneum device 5051 feeds gas into a body cavity of the patient 5071 through the insufflation tube 5019 to inflate the body cavity in order to secure the visual field of the endoscope 5001 and secure the working space for the surgeon 5067. A recorder 5053 is a device capable of recording various types of information associated with surgery. A printer 5055 is a device capable of printing various types of information associated with surgery in various forms such as text, image, graph, or the like.

<1-1-2. Detailed Configuration Example of Support Arm Device>

An example of a detailed configuration of the support arm device 5027 according to the present embodiment will be described with reference to FIG. 1.

The support arm device 5027 includes the base unit 5029 which is a pedestal, and the arm unit 5031 extending from the base unit 5029. In the example of FIG. 1, the arm unit 5031 is formed with the multiple joints 5033a, 5033b, and 5033c and the multiple links 5035a and 5035b coupled via the joints 5033b. However, for the sake of simplicity, FIG. 1 illustrates the configuration of the arm unit 5031 in a simplified manner. In practice, the shapes, the number and the arrangement of the joints 5033a to 5033c and the links 5035a and 5035b, the directions of the rotation axes of the joints 5033a to 5033c, or the like, can be appropriately set so that the arm unit 5031 has a desired degree of freedom. For example, the arm unit 5031 can be suitably configured to have six degrees of freedom, or more. With this configuration, the endoscope 5001 can be freely moved within the movable range of the arm unit 5031, making it possible to insert the lens barrel 5003 of the endoscope 5001 into the body cavity of the patient 5071 from a desired direction.

Each of the joints 5033a to 5033c is equipped with an actuator. Each of the joints 5033a to 5033c is rotatable about a predetermined rotation axis by the drive of the actuator. The drive of the actuator is controlled by the arm control device 5045, thereby controlling the rotation angle of each of the joints 5033a to 5033c and controlling the drive of the arm unit 5031. This control can achieve the control of the position and posture of the endoscope 5001. At this time, the arm control device 5045 can control the drive of the arm unit 5031 by various known control methods such as force control or position control.

For example, the surgeon 5067 may appropriately perform an operation input via the input device 5047 (including the foot switch 5057) so as to appropriately control the drive of the arm unit 5031 by the arm control device 5045 in accordance with the operation input, leading to the control of the position and posture of the endoscope 5001. With this control, it is possible to move the endoscope 5001 on the distal end of the arm unit 5031 from a certain position to another certain position, and thereafter fixedly support the endoscope 5001 at a new position after the movement. Incidentally, the arm unit 5031 may be operated by a method referred to as a master-slave method. In this case, the arm unit 5031 (slave) can be remotely operated by the user via the input device 5047 (master console) installed at a place away from the operating room or in the operating room.

Furthermore, in a case where the force control is applied, the arm control device 5045 may perform power assist control, in which after receiving an external force from the user, the actuators of the individual joints 5033a to 5033c are driven so as to smoothly move the arm unit 5031 in accordance with the external force. With this control, it is possible to move the arm unit 5031 with a relatively light force when the user moves the arm unit 5031 while directly touching the arm unit 5031. This makes it possible to further intuitively move the endoscope 5001 with simpler operation, leading to enhancement of convenience for the user.

Here, the endoscope 5001 is typically supported by a doctor as an endoscopist in endoscopic surgery. In contrast, the use of the support arm device 5027 makes it possible to reliably secure the position of the endoscope 5001 without manual work, leading to stable acquisition of an image of the surgical site and smooth execution of surgery.

Note that the arm control device 5045 does not necessarily have to be provided in the cart 5037. Furthermore, the arm control device 5045 does not necessarily have to be one device. For example, the arm control device 5045 may be provided in each of the joints 5033a to 5033c of the arm unit 5031 of the support arm device 5027, and the multiple arm control devices 5045 may cooperate with each other to achieve the drive control of the arm unit 5031.

<1-1-3. Detailed Configuration Example of Light Source Device>

An example of a detailed configuration of the light source device 5043 according to the present embodiment will be described with reference to FIG. 1.

The light source device 5043 supplies the endoscope 5001 with irradiation light for imaging the surgical site. The light source device 5043 is formed with, for example, an LED, a laser light source, or a white light source constituted by a combination of these. At this time, in a case where the white light source is constituted with the combination of RGB laser light sources, it is possible to control the output intensity and the output timing of individual colors (individual wavelengths) with high accuracy. Accordingly, it is possible to perform white balance adjustment of the captured image on the light source device 5043. Furthermore, in this case, by emitting the laser light from each of the RGB laser light sources to an observation target on the time-division basis and by controlling the drive of the imaging element of the camera head 5005 in synchronization with the light emission timing, it is also possible to capture the image corresponding to each of RGB colors on the time-division basis. According to this method, a color image can be obtained even if color filters are not provided for the imaging element.

Furthermore, the drive of the light source device 5043 may be controlled so as to change the intensity of the output light at predetermined time intervals. With the control of the drive of the imaging element of the camera head 5005 in synchronization with the timing of the change of the intensity of the light so as to obtain images on the time-division basis and combine the images, it is possible to generate an image with high dynamic range without a state such as blackout shadows or blown out highlights (overexposure).

Furthermore, the light source device 5043 may be configured to be able to supply light in a predetermined wavelength band corresponding to special light observation. The special light observation is used to perform narrowband light observation (narrow band imaging). The narrowband light observation uses the wavelength dependency of the light absorption in the body tissue and emits light in a narrower band compared with the irradiation light (that is, white light) at normal observation, thereby imaging a predetermined tissue such as a blood vessel of the mucosal surface layer with high contrast. Alternatively, the special light observation may include fluorescence observation to obtain an image by fluorescence generated by emission of excitation light. Fluorescence observation can be performed to observe fluorescence emitted from a body tissue to which excitation light is applied (autofluorescence observation), and can be performed with local administration of reagent such as indocyanine green (ICG) to the body tissue, and together with this, excitation light corresponding to the fluorescence wavelength of the reagent is emitted to the body tissue to obtain a fluorescent image, or the like. The light source device 5043 can be configured to be able to supply narrow band light and/or excitation light corresponding to such special light observation.

<1-1-4. Detailed Configuration Example of Camera Head and CCU>

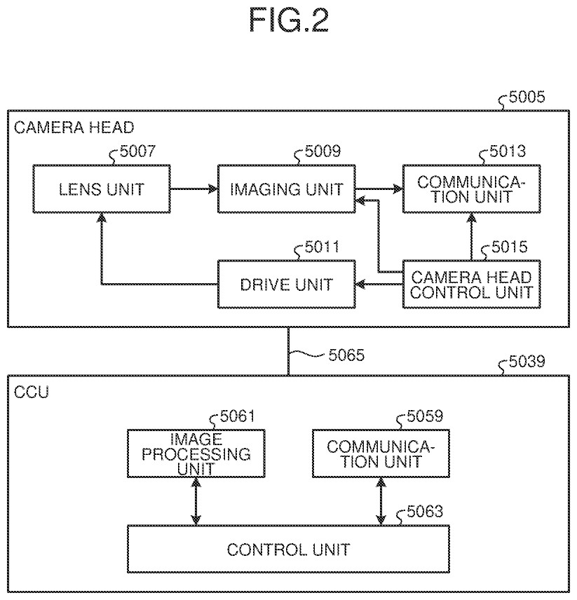

An example of a detailed configuration of the camera head 5005 and the CCU 5039 of the endoscope 5001 will be described with reference to FIG. 2. FIG. 2 is a block diagram illustrating an example of a detailed configuration of the camera head 5005 and the CCU 5039 in FIG. 1.

As illustrated in FIG. 2, the camera head 5005 includes, as functional configuration, a lens unit 5007, an imaging unit 5009, a drive unit 5011, a communication unit 5013, and a camera head control unit 5015. Furthermore, the CCU 5039 includes, as a functional configuration, a communication unit 5059, an image processing unit 5061, and a control unit 5063. The camera head 5005 and the CCU 5039 are connected with each other by a transmission cable 5065 so as to enable bi-directional communication.

First, the functional configuration of the camera head 5005 will be described. The lens unit 5007 is an optical system provided at a connecting portion with the lens barrel 5003. The observation light captured from the distal end of the lens barrel 5003 is guided to the camera head 5005 so as to be incident on the lens unit 5007. The lens unit 5007 is formed by a combination of multiple lenses including a zoom lens and a focus lens. The optical characteristics of the lens unit 5007 are adjusted so as to focus the observation light on a light receiving surface of the imaging element of the imaging unit 5009. In addition, the zoom lens and the focus lens are configured to be movable in position on the optical axis in order to adjust the magnification and the focal point of the captured image.

The imaging unit 5009 includes an imaging element and is arranged at a subsequent stage of the lens unit 5007. The observation light having passed through the lens unit 5007 is focused on the light receiving surface of the imaging element, and an image signal corresponding to the observation image is generated by photoelectric conversion. The image signal generated by the imaging unit 5009 is supplied to the communication unit 5013.

An example of the imaging element constituting the imaging unit 5009 is a complementary metal oxide semiconductor (CMOS) image sensor capable of color photography with Bayer arrays. Note that the imaging element may be an imaging element compatible with imaging of a high resolution image of 4K or more. With acquisition of the image of the surgical site with high resolution, the surgeon 5067 can grasp the states of the surgical site in more detail, leading to smooth progress of the surgery.

In addition, the imaging element constituting the imaging unit 5009 includes a pair of imaging elements for acquiring image signals for the right eye and the left eye corresponding to 3D display. With implementation of 3D display, the surgeon 5067 can grasp the depth of the living tissue in the surgical site with higher accuracy. When the imaging unit 5009 is a multi-plate type, multiple lens units 5007 is also provided corresponding to each of the imaging elements.

Furthermore, the imaging unit 5009 does not necessarily have to be provided on the camera head 5005. For example, the imaging unit 5009 may be provided inside the lens barrel 5003 immediately behind the objective lens.

The drive unit 5011 includes an actuator and moves the zoom lens and the focus lens of the lens unit 5007 by a predetermined distance along the optical axis under the control of the camera head control unit 5015. With this operation, the magnification and focal point of the image captured by the imaging unit 5009 can be appropriately adjusted.

The communication unit 5013 includes a communication device for transmitting and receiving various types of information to and from the CCU 5039. The communication unit 5013 transmits the image signal obtained from the imaging unit 5009 as RAW data to the CCU 5039 via the transmission cable 5065. At this time, in order to display the captured image of the surgical site with low latency, the image signal is preferably transmitted by optical communication. This is because, at the time of surgery the surgeon 5067 performs surgery while observing the condition of the affected part using captured images, and thus displaying moving images of the surgical site in real time as much as possible is demanded for safer and more reliable surgery. In a case where optical communication is performed, the communication unit 5013 is provided with a photoelectric conversion module that converts an electric signal into an optical signal. The image signal is converted into an optical signal by the photoelectric conversion module and then transmitted to the CCU 5039 via the transmission cable 5065.

Furthermore, the communication unit 5013 receives a control signal for controlling drive of the camera head 5005 from the CCU 5039. The control signal includes information associated with imaging conditions, such as information designating a frame rate of a captured image, information designating an exposure value at the time of imaging, and/or information designating the magnification and focal point of the captured image. The communication unit 5013 supplies the received control signal to the camera head control unit 5015. Note that the control signal from the CCU 5039 may also be transmitted by optical communication. In this case, the communication unit 5013 is provided with a photoelectric conversion module that converts an optical signal into an electric signal, and the control signal is converted into an electric signal by the photoelectric conversion module and then supplied to the camera head control unit 5015.

Note that the imaging conditions such as the frame rate, the exposure value, the magnification, and the focus are automatically set by the control unit 5063 of the CCU 5039 on the basis of the acquired image signal. That is, an Auto Exposure (AE) function, an Auto Focus (AF) function, and an Auto White Balance (AWB) function are to be installed in the endoscope 5001.

The camera head control unit 5015 controls the drive of the camera head 5005 on the basis of the control signal from the CCU 5039 received via the communication unit 5013. For example, the camera head control unit 5015 controls drive of the imaging element of the imaging unit 5009 on the basis of information designating the frame rate of the captured image and/or information designating exposure at the time of imaging. Furthermore, for example, the camera head control unit 5015 appropriately moves the zoom lens and the focus lens of the lens unit 5007 via the drive unit 5011 on the basis of the information designating the magnification and the focal point of the captured image. The camera head control unit 5015 may further include a function of storing information for identifying the lens barrel 5003 and the camera head 5005.

Note that arranging the lens unit 5007, the imaging unit 5009, or the like, in a hermetically sealed structure having high airtightness and waterproofness would make it possible to allow the camera head 5005 to have resistance to autoclave sterilization processing.

Next, a functional configuration of the CCU 5039 will be described. The communication unit 5059 includes a communication device for transmitting and receiving various types of information to and from the camera head 5005. The communication unit 5059 receives an image signal transmitted from the camera head 5005 via the transmission cable 5065. At this time, as described above, the image signal can be suitably transmitted by optical communication. In this case, for optical communication, the communication unit 5059 is provided with a photoelectric conversion module that converts an optical signal into an electric signal. The communication unit 5059 supplies the image signal converted into the electric signal to the image processing unit 5061.

Furthermore, the communication unit 5059 transmits a control signal for controlling the drive of the camera head 5005 to the camera head 5005. The control signal may also be transmitted by optical communication.

The image processing unit 5061 performs various types of image processing on the image signal in RAW data transmitted from the camera head 5005. Examples of the image processing include various known signal processing such as development processing, high image quality processing (band enhancement processing, super-resolution processing, Noise Reduction (NR) processing, camera shake correction processing, and/or the like), and/or enlargement processing (electronic zoom processing). Furthermore, the image processing unit 5061 performs demodulation processing on the image signals for performing AE, AF, and AWB.

The image processing unit 5061 includes a processor such as a CPU and a GPU. The processor operates in accordance with a predetermined program to enable execution of the above-described image processing and demodulation processing. Note that, in a case where the image processing unit 5061 includes multiple GPUs, the image processing unit 5061 appropriately divides the information related to image signals, and performs image processing in parallel by the multiple GPUs.

The control unit 5063 performs various types of control related to imaging of the surgical site by the endoscope 5001 and display of the captured image. For example, the control unit 5063 generates a control signal for controlling the drive of the camera head 5005. At this time, in a case where the imaging condition has been input by the user, the control unit 5063 generates the control signal on the basis of the input by the user. Alternatively, in a case where the endoscope 5001 includes the AE function, the AF function, and the AWB function, the control unit 5063 appropriately calculates the optimum exposure value, a focal length, and white balance in accordance with a result of demodulation processing performed by the image processing unit 5061, and generates a control signal.

Furthermore, the control unit 5063 controls the display device 5041 to display the image of the surgical site on the basis of the image signal that has undergone image processing performed by the image processing unit 5061. At this time, the control unit 5063 recognizes various objects in the image of the surgical site by using various image recognition techniques. For example, the control unit 5063 detects the shape, color, or the like of the edge of an object included in the surgical site image, making it possible to recognize a surgical tool such as forceps, a specific living body site, bleeding, occurrence of mist at the time of using the energy treatment tool 5021, or the like. When displaying the image of the operation site on the display device 5041, the control unit 5063 superimposes and displays various surgical operation assistance information on the image of the surgical site by using the recognition result. Surgical assistance information is superimposed and displayed, and presented to the surgeon 5067, thereby making it possible to proceed with surgery more safely and reliably.

The transmission cable 5065 connecting the camera head 5005 and the CCU 5039 is an electric signal cable compatible with electric signal communication, an optical fiber compatible with optical communication, or a composite cable of these.

Here, while FIG. 2 is an example in which wired communication is performed using the transmission cable 5065, the communication between the camera head 5005 and the CCU 5039 may be performed wirelessly. In a case where the communication between the two units is performed wirelessly, there is no need to dispose the transmission cable 5065 in the operating room, making it possible to eliminate a situation in which the movement of the medical workers in the operating room is hindered by the transmission cable 5065.

<1-1-5. Example of External Configuration of Support Arm Device>

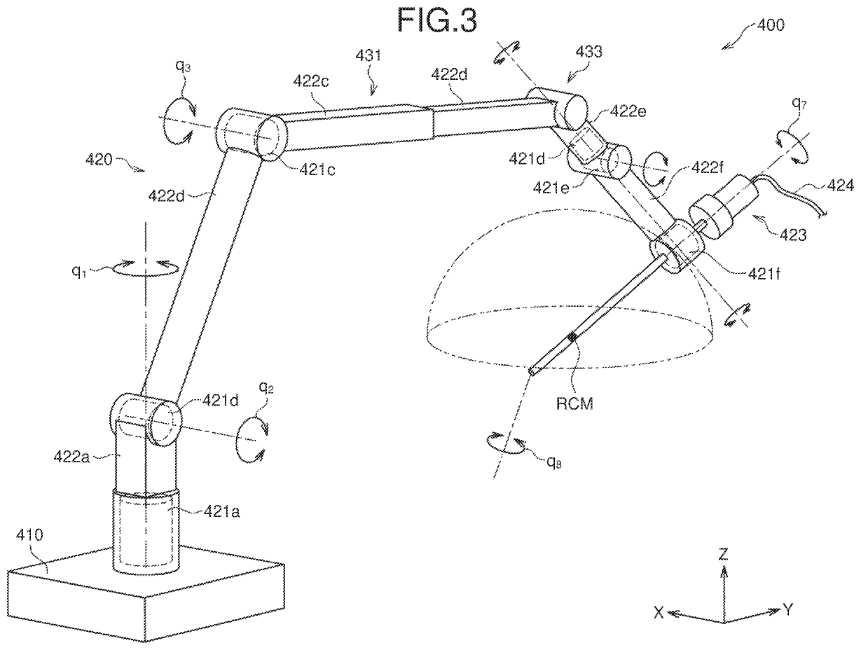

An example of an external configuration of a support arm device 400 according to the present embodiment will be described with reference to FIG. 3. FIG. 3 is a diagram illustrating an example of an external configuration of the support arm device 400 according to the present embodiment. The support arm device 400 corresponds to the support arm device 5027 described above.

As illustrated in FIG. 3, the support arm device 400 according to the present embodiment includes a base unit 410 and an arm unit 420. The base unit 410 is a base of the support arm device 400, and the arm unit 420 extends from the base unit 410. Furthermore, although not illustrated in FIG. 3, a control unit that integrally controls the support arm device 400 may be provided in the base unit 410, and the drive of the arm unit 420 may be controlled by the control unit. The control unit includes various signal processing circuits such as a CPU and a DSP, for example.

The arm unit 420 includes multiple active joints 421a to 421f, multiple links 422a to 422f, and an endoscope device 423 as a distal end unit provided at the distal end of the arm unit 420. The links 422a to 422f are substantially rod-shaped members. One end of the link 422a is coupled to the base unit 410 via the active joint 421a, the other end of the link 422a is coupled to one end of the link 422b via the active joint 421b, and the other end of the link 422b is coupled to one end of the link 422c via the active joint 421c. The other end of the link 422c is coupled to the link 422d via a passive slide mechanism 431, and the other end of the link 422d is coupled to one end of the link 422e via a passive joint 433. The other end of the link 422e is coupled to one end of the link 422f via the active joints 421d and 421e. The endoscope device 423 is coupled to the distal end of the arm unit 420, that is, the other end of the link 422f via the active joint 421f. In this manner, the ends of the multiple links 422a to 422f are coupled to each other by the active joints 421a to 421f, the passive slide mechanism 431, and the passive joints 433 with the base unit 410 as a fulcrum, thereby forming an arm shape extending from the base unit 410.

The drive control of the actuators provided in the individual active joints 421a to 421f in such an arm unit 420 is performed, thereby controlling the position and posture of the endoscope device 423. In the present embodiment, the distal end of the endoscope device 423 enters the body cavity of the patient, which is the operation site, and captures a partial region of the surgical site. However, the distal end unit provided at the distal end of the arm unit 420 is not limited to the endoscope device 423, and various medical instruments may be connected to the distal end of the arm unit 420 as the distal end unit. In this manner, the support arm device 400 according to the present embodiment is configured as a medical support arm device including a medical instrument.

Hereinafter, the support arm device 400 will be described by defining coordinate axes as illustrated in FIG. 3. Furthermore, the up-down direction, the front-rear direction, and the left-right direction are defined in accordance with the coordinate axes. That is, the up-down direction with respect to the base unit 410 installed on the floor surface is defined as the z-axis direction and the up-down direction. Furthermore, a direction orthogonal to the z-axis and in which the arm unit 420 extends from the base unit 410 (that is, the direction in which the endoscope device 423 is located with respect to the base unit 410) is defined as a y-axis direction and a front-rear direction. Furthermore, a direction orthogonal to the y-axis and the z-axis is defined as an x-axis direction and a left-right direction.

The active joints 421a to 421f pivotably couple the links to each other. The active joints 421a to 421f have actuators, and have a rotation mechanism that is rotationally driven about a predetermined rotation axis by drive of the actuators. By controlling the rotational drive of each of the active joints 421a to 421f, it is possible to control the drive of the arm unit 420, such as extending or contracting (folding) of the arm unit 420, for example. Here, the drive of the active joints 421a to 421f can be controlled by known whole-body cooperative control and idealized joint control, for example. Since the active joints 421a to 421f have the rotation mechanism as described above, the drive control of the active joints 421a to 421f in the following description specifically means the control of the rotation angles and/or generated torques in the active joints 421a to 421f (torques generated by the active joints 421a to 421f).

The passive slide mechanism 431 is an aspect of a passive mode change mechanism, and couples the link 422c and the link 422d so as to be movable forward/backward in a predetermined direction. For example, the passive slide mechanism 431 may couple the link 422c and the link 422d to each other so as to be linearly movable. However, the forward/backward movement of the link 422c and the link 422d is not limited to the linear movement, and may be a forward/backward movement in a direction forming an arc shape. The passive slide mechanism 431 is operated to move forward/backward by a user, for example, and makes a distance between the link 422c on one end side of the active joint 421c and the passive joint 433 variable. This makes it possible to change the overall mode of the arm unit 420.

The passive joint 433 is an aspect of the passive mode change mechanism, and pivotably couple the link 422d and the link 422e to each other. Having received a pivot operation from the user, the passive joint 433 makes the angle formed by the link 422d and the link 422e variable. This makes it possible to change the overall mode of the arm unit 420.

As a specific example, the “posture of the arm unit” indicate the state of the arm unit that can be changed by the drive control of the actuators provided in the active joints 421a to 421f by the control unit in a state where the distance between the active joints adjacent to each other across one or multiple links is constant. In the present disclosure, the “posture of the arm unit” is not limited to the state of the arm unit that can be changed by the drive control of the actuator. For example, the “posture of the arm unit” may be a state of the arm unit, which has been changed by cooperative operation of the joints. Furthermore, in the present disclosure, the arm unit does not necessarily have to include a joint. In this case, the “posture of the arm unit” represents a position with respect to a target or a relative angle with respect to the target. Moreover, “the mode of the arm unit” indicates the state of the arm unit that can change together with the change in the distance between the active joints adjacent to each other across the link(s) or the angle formed by the links joining the active joints adjacent to each other, along with the operation of the passive mode change mechanism, can correspond to the “form of the arm unit”. In the present disclosure, the “mode of the arm unit” is not limited to the state of the arm unit that can change together with the change in the distance between the active joints adjacent to each other across the link or the angle formed by the links joining the active joints adjacent to each other. For example, the “mode of the arm unit” may be a state of the arm unit that can change together with the change in a positional relationship or angles between the joints by cooperative operations of the joints. Furthermore, when the arm unit does not include joints, the “mode of the arm unit” may be a state of the arm unit that can change together with the change in the position with respect to the target or the relative angle with respect to the target.

The support arm device 400 according to the present embodiment includes six active joints, namely, the active joints 421a to 421f, achieving six degrees of freedom regarding the drive of the arm unit 420. That is, while the drive control of the support arm device 400 is actualized by the drive control of the six active joints 421a to 421f by the control unit, the passive slide mechanism 431 and the passive joint 433 are not defined as the target of the drive control by the control unit.

Specifically, as illustrated in FIG. 3, the active joints 421a, 421d, and 421f are arranged such that the longitudinal direction of each of the connected links 422a and 422e and the imaging direction of the connected endoscope device 423 are aligned with the rotation axis direction. The active joints 421b, 421c, and 421e are arranged such that the x-axis direction, which is a direction in which the coupling angle of each of the connected links 422a to 422c, 422e, and 422f and the endoscope device 423 is changed in a y-z plane (plane defined by the y-axis and the z-axis), is aligned with the rotation axis direction. In this manner, in the present embodiment, the active joints 421a, 421d, and 421f have a function of performing a motion referred to as yawing, and the active joints 421b, 421c, and 421e have a function of performing a motion referred to as pitching.

With such a configuration of the arm unit 420, the support arm device 400 according to the present embodiment can achieve six degrees of freedom in the drive of the arm unit 420, making it possible to freely move the endoscope device 423 within a movable range of the arm unit 420. FIG. 3 illustrates a hemisphere as an example of a movable range of the endoscope device 423. Assuming that the remote center of motion (RCM) in the hemisphere is an imaging center of the surgical site to be imaged by the endoscope device 423, the surgical site can be imaged from various angles by moving the endoscope device 423 on the spherical surface of the hemisphere in a state where the imaging center of the endoscope device 423 is fixed to the center point of the hemisphere.

Although the arm unit 420 of the support arm device 400 has been described as having multiple joints and having six degrees of freedom, the present disclosure is not limited to these. Specifically, the arm unit 420 is only required to have a structure in which the endoscope 5001 or an exoscope can be disposed at the distal end. For example, the arm unit 420 may have a configuration having only one degree of freedom to allow the endoscope 5001 to drive so as to move forward in a direction of entering the body cavity of the patient and a direction of moving backward.

An example of the endoscopic surgery system 5000 to which the technique according to the present disclosure can be applied has been described above. Although the endoscopic surgery system 5000 has been described here as an example, the system to which the technique according to the present disclosure can be applied is not limited to such an example. For example, the technique according to the present disclosure may be applied to a flexible endoscope surgery system for examination or a microscopic surgery system.

<1-2. Configuration of Medical Observation System>

<1-2-1. Schematic Configuration Example of Medical Observation System>

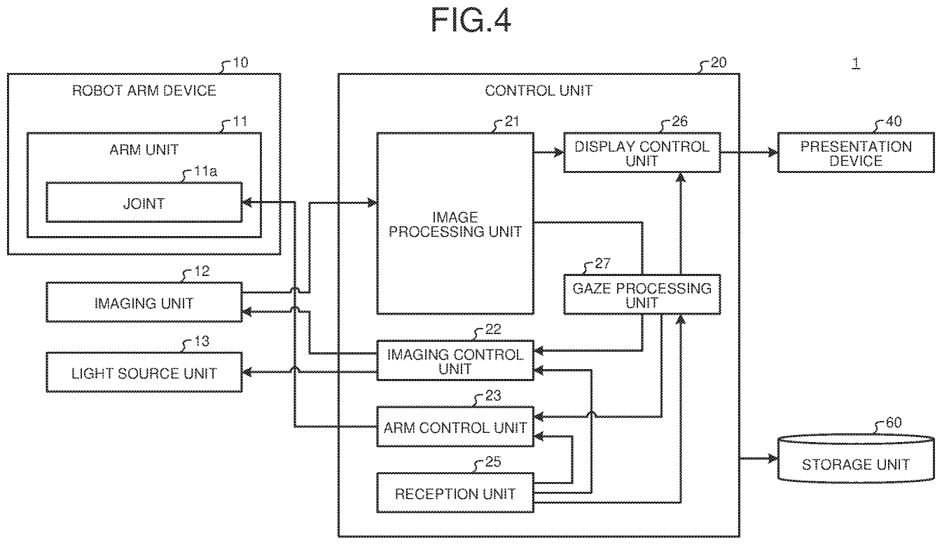

An example of a schematic configuration of a medical observation system 1 according to the present embodiment will be described with reference to FIG. 4. FIG. 4 is a diagram illustrating an example of a schematic configuration of the medical observation system 1 according to the present embodiment. The medical observation system 1 according to the present embodiment is a system that can be combined with the endoscopic surgery system 5000 described above.

As illustrated in FIG. 4, the medical observation system 1 includes a robot arm device 10 (corresponding to the support arm device 5027), an imaging unit 12 (corresponding to the endoscope 5001), a light source unit 13 (corresponding to the light source device 5043), a control unit 20 (corresponding to the CCU 5039), a presentation device 40 (corresponding to the display device 5041), and a storage unit 60. Hereinafter, each functional unit included in the medical observation system 1 will be described.

First, before describing the details of the configuration of the medical observation system 1, an outline of processing of the medical observation system 1 will be described. In the medical observation system 1, for example, the imaging unit 12 is inserted into the body of the patient through a medical puncture device referred to as a trocar, and the surgeon 5067 performs the laparoscopic surgery while capturing an image of an area of interest. At this time, by driving the robot arm device 10, the imaging unit 12 can freely change the image capturing position.

Specifically, the medical observation system 1 images the inside of the abdominal cavity of the patient by the imaging unit 12 to recognize the environment inside the abdominal cavity, and drives the robot arm device 10 on the basis of the recognition result of the environment inside the abdominal cavity. Here, the imaging range in the abdominal cavity changes by driving the robot arm device 10. When the imaging range in the abdominal cavity has changed, the medical observation system 1 recognizes the changed environment and drives the robot arm device 10 on the basis of the recognition result. The medical observation system 1 repeats image recognition of the environment in the abdominal cavity and driving of the robot arm device 10. That is, the medical observation system 1 executes processing combining image recognition processing and processing of controlling the position and posture of the robot arm device 10.

(Robot Arm Device 10)

The robot arm device 10 includes an arm unit 11 (corresponding to the arm unit 5031) that is a multilink structure including multiple joints and multiple links, and drives the arm unit within a movable range to control the position and posture of a distal end unit provided at the distal end of the arm unit 11 which is an articulated arm.

In the robot arm device 10 according to the present embodiment, the electronic degree of freedom of changing the gaze line by cutting out the captured image (wide angle/cutout function) and the degree of freedom obtained by the actuator of the arm unit 11 are all treated as the degrees of freedom of the robot. This makes it possible to achieve execution of motion control that links the electronic degree of freedom of changing the gaze line and the degree of freedom of the joint obtained by the actuator.

Specifically, the arm unit 11 is a multilink structure including multiple joints and multiple links, and its driving is controlled by the control made by an arm control unit 23 to be described below. In FIG. 4, multiple joints is represented as one joint 11a. Specifically, the joint 11a pivotably couples the links in the arm unit 11, and drives the arm unit 11 by controlling the rotational drive of the joint 11a under the control of the arm control unit 23. Furthermore, in order to obtain information regarding the position and posture of the arm unit 11, the arm unit 11 may include motion sensors (not illustrated) including an acceleration sensor, a gyro sensor, and a geomagnetic sensor.

(Imaging unit 12)

The imaging unit 12 is provided at the distal end of the arm unit (medical arm) 11, and captures images of various imaging targets. That is, the arm unit 11 supports the imaging unit 12. As described above, the imaging unit 12 may be, for example, a stereo endoscope, an oblique-viewing endoscope (not illustrated), a forward straight-viewing endoscope (not illustrated), an endoscope with a multi-direction simultaneous imaging function (not illustrated), or a microscope, and is not particularly limited.

Furthermore, the imaging unit 12 captures, for example, an operative field image including various medical instruments, organs, and the like in the abdominal cavity of the patient. Specifically, the imaging unit 12 is a camera or the like capable of capturing an image capture target in a form of a moving image or a still image. More specifically, the imaging unit 12 is a wide-angle camera including a wide-angle optical system. For example, while the viewing angle of a normal endoscope is about 80°, the viewing angle of the imaging unit 12 according to the present embodiment may be 140°. Note that the viewing angle of the imaging unit 12 may be smaller than 140° or may be 140° or more as long as it exceeds 80°. The imaging unit 12 transmits an electric signal (pixel signal) corresponding to the captured image to the control unit 20. Furthermore, the arm unit 11 may support a medical instrument such as the forceps 5023.

Furthermore, in the present embodiment, a stereo endoscope capable of distance measurement may be used as the imaging unit 12. Moreover, a depth sensor (distance measuring device) (not illustrated) may be provided separately from the imaging unit 12 using an endoscope other than the stereo endoscope. In this case, the imaging unit 12 may be a monocular endoscope. The depth sensor may be, for example, a sensor that performs distance measurement using a time of flight (ToF) method in which distance measurement is performed using a return time of reflection of pulsed light from a subject or using a structured light method in which distance measurement is performed by distortion of a pattern of emitted lattice-shaped pattern light. Alternatively, in the present embodiment, the imaging unit 12 itself may be provided with a depth sensor. In this case, the imaging unit 12 can perform distance measurement by the ToF method simultaneously with imaging. Specifically, the imaging unit 12 includes multiple light receiving elements (not illustrated), and can generate an image or calculate distance information on the basis of a pixel signal obtained from the light receiving elements.

(Light Source Unit 13)

The light source unit 13 emits light to the imaging target of the imaging unit 12. The light source unit 13 can be actualized by a wide-angle lens light emitting diode (LED), for example. For example, the light source unit 13 may be configured by combining a normal LED and a lens so as to diffuse light. Furthermore, the light source unit 13 may have a configuration in which light transmitted through an optical fiber (light guide) is diffused (widened) by a lens. In addition, the light source unit 13 may expand the light emission range by emitting light by directing the optical fiber itself in multiple directions.

(Control Unit 20)

The control unit 20 mainly includes an image processing unit 21, an imaging control unit 22, an arm control unit 23, a reception unit 25, a display control unit 26, and a gaze processing unit 27. The control unit 20 is actualized by execution of programs stored in the storage unit 60 (for example, information processing program according to the embodiment of the present disclosure) by a central processing unit (CPU), a micro processing unit (MPU), or the like, using random access memory (RAM) or the like, as a working area. In addition, the control unit 20 is a controller and may be implemented by, for example, an integrated circuit such as an application specific integrated circuit (ASIC) or a field programmable gate array (FPGA). The control unit 20 corresponds to an information processing device.

The image processing unit 21 executes various types of processing on the imaging target captured by the imaging unit 12. Specifically, the image processing unit 21 acquires an image of the imaging target captured by the imaging unit 12, and generates various images on the basis of the image captured by the imaging unit 12. More specifically, the image processing unit 21 can generate an image by cutting out and enlarging a display target region (cutout range) of the image captured by the imaging unit 12. In this case, for example, the image processing unit 21 may change an image cutout position (cutout range) according to a condition such as the state of the image captured by the imaging unit 12.

The imaging control unit 22 controls the imaging unit 12. For example, the imaging control unit 22 controls the imaging unit 12 to image the operative field. The imaging control unit 22 controls, for example, an enlargement magnification of the imaging unit 12. Furthermore, for example, the imaging control unit 22 may control the enlargement magnification of the imaging unit 12 on the basis of the input information received by the reception unit 25, or may control the enlargement magnification of the imaging unit 12 according to the state of the image captured by the imaging unit 12, the display state, or the like. Furthermore, the imaging control unit 22 may control the focus (focal length) of the imaging unit 12 or may control the gain (sensitivity) of the imaging unit 12 (specifically, the image sensor of the imaging unit 12) according to the state of the image captured by the imaging unit 12 or the like.

Furthermore, the imaging control unit 22 controls the light source unit 13. For example, the imaging control unit 22 controls the brightness of the light source unit 13 when the imaging unit 12 images the operative field. For example, the imaging control unit 22 controls the brightness of the light source unit 13 on the basis of the input information received by the reception unit 25. The surgeon 5067 operates the input device 5047 to input the input information.

The arm control unit 23 integrally controls the robot arm device 10 and controls driving of the arm unit 11. Specifically, the arm control unit 23 controls the driving of the joint 11a so as to control the driving of the arm unit 11. More specifically, by controlling the amount of current supplied to the motor in the actuator of the joint 11a, the arm control unit 23 controls the number of rotations of the motor and controls the rotation angle and the generated torque in the joint 11a. For example, the arm control unit 23 can autonomously control the position and posture (for example, the angle) of the arm unit 11 according to information such as the input information received by the reception unit 25 and the information based on the image captured by the imaging unit 12.

The reception unit 25 can receive input information input from the input device 5047 and various input information (sensing data) from other devices (for example, a depth sensor or the like) and can output the input information to the imaging control unit 22 and the arm control unit 23. The input information may be a magnification rate of the imaging unit 12 or instruction information for changing the position/posture of the arm unit 11, for example.

The display control unit 26 causes the presentation device 40 to display various images. For example, the display control unit 26 controls to output a wide-angle image (first operative field image), a cutout image (second operative field image), and the like generated by the image processing unit 21 to the presentation device 40 to display.

From the image (for example, a wide-angle image) input from the image processing unit 21, the gaze processing unit 27 determines the position and posture of the imaging unit 12 optimizing the tracking and image cutout of the gaze target (for example, an instrument, an organ, or the like). For example, the gaze processing unit 27 extracts a gaze target portion, obtains a gaze point of the gaze target, and generates gaze point information related to the gaze point (for example, information such as the position of the gaze point and a requested gaze line vector related to the gaze point). Furthermore, the gaze processing unit 27 obtains the movable range (endoscope movable range) of the imaging unit 12 on the basis of the gaze point information, determines the position and posture, the cutout visual field, and the like of the imaging unit 12 from the movable range information, and generates posture information related to the position and posture, the cutout visual field, and the like of the imaging unit 12. This posture information is transmitted to, for example, the imaging control unit 22, the arm control unit 23, the display control unit 26, and the like.

### (Presentation Device 40)

The presentation device 40 displays various images. The presentation device 40 displays an image captured by the imaging unit 12, for example. The presentation device 40 can be, for example, a display including a liquid crystal display (LCD), an organic electro-luminescence (EL) display, or the like. The presentation devices 40 may be provided in plurality according to the application.

### (Storage Unit 60)

The storage unit 60 stores various types of information. The storage unit 60 is implemented by semiconductor memory elements such as random access memory (RAM) and flash memory, or other storage devices such as a hard disk or an optical disc.

<1-2-2. Detailed Configuration Example of Robot Arm Device>

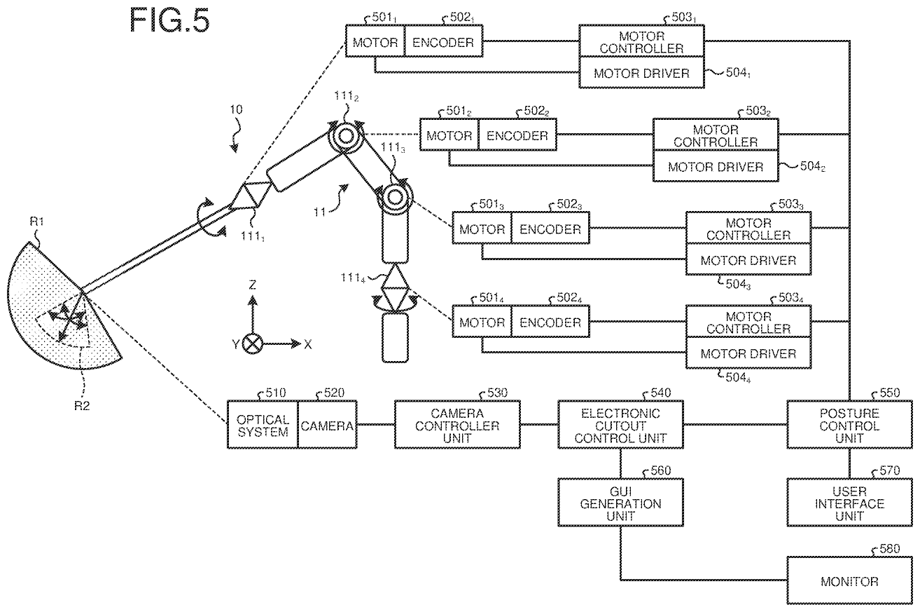

An example of a detailed configuration of the robot arm device 10 according to the present embodiment will be described with reference to FIG. 5. FIG. 5 is a diagram illustrating an example of a detailed configuration of the robot arm device 10 according to the present embodiment.

As illustrated in FIG. 5, the arm unit 11 of the robot arm device 10 includes a first joint 111₁, a second joint 111₂, a third joint 111₃, and a fourth joint 111₄. The robot arm device 10 is connected to a camera control unit 530 (corresponding to the imaging control unit 22), an electronic cutout control unit 540 (corresponding to the image processing unit 21), a posture control unit 550 (corresponding to the arm control unit 23), a GUI generation unit 560 (corresponding to a display control unit 24), a user interface unit 570 (corresponding to the input device 5047), and a monitor 580 (corresponding to the presentation device 40).

The first joint 111₁ includes a motor 501₁, an encoder 502₁, a motor controller 503₁, and a motor driver 504₁. Since the second joint 111₂ to the fourth joint 111₄ also have the configuration similar to the first joint 111₁, the first joint 111₁ will be described below as an example.

The motor 501₁ is driven under the control of the motor driver 504₁ to drive the first joint 111₁. The motor 501₁ drives the first joint 111₁ in a direction of an arrow attached to the first joint 111₁, for example. The motor 501₁ drives the first joint 111₁ to control the position and posture of the arm unit 11 and the position and posture of the lens barrel (corresponding to an optical system 510) and a camera 520 (corresponding to the camera head 5005). In the present embodiment, it is also allowable, as an embodiment of the endoscope, to provide the camera 520 (corresponding to the lens unit 5007 and the imaging unit 5009, in this case) at the distal end of the lens barrel. Under the control of and the motor controller 503₁, the encoder 502₁ detects information related to the rotation angle of the first joint 111₁. That is, the encoder 502₁ acquires information related to the posture of the first joint 111₁.

The optical system 510 is a wide-angle optical system including a wide-angle lens, for example. The camera 520 captures an image capture target such as an organ of a patient or a medical instrument used for treatment, for example. As will be described below, in the present embodiment, for example, a display target region R2 desired by the user in a wide-angle visual field R1 is cut out to generate a cutout image (second operative field image).

The camera control unit 530 corresponds to the CCU 5039 illustrated in FIG. 2. That is, the camera control unit 530 integrally controls operations of imaging processing by the camera 520 and processing of video to be displayed on the monitor 580.

The electronic cutout control unit 540 cuts out a predetermined region from the video obtained by imaging the image capture target received from the camera control unit 530, and outputs the video of the region to the GUI generation unit 560. Processing of cutting out a predetermined region from the video obtained by imaging the image capture target will be described below.

The GUI generation unit 560 generates video data obtained by performing various types of processing on the video cut out from the electronic cutout control unit 540, and outputs the generated video data to the monitor 580. This allows the monitor 580 to display various videos generated by the GUI generation unit 560. Note that part or both of the electronic cutout control unit 540 and the GUI generation unit 560 may be provided in the camera control unit 530.

The posture control unit 550 controls the position and posture of the arm unit 11. Specifically, the posture control unit 550 controls the motor controller 503₁ to 503₄, the motor driver 504₁ to 504₄, and the like to respectively control the first joint 111₁ to the fourth joint 111₄. This allows the posture control unit 550 to control the position and posture of the arm unit 11. The posture control unit 550 may be included in the camera control unit 530.

The user interface unit 570 receives various operations from the user. The user interface unit 570 receives an operation for controlling the position and posture of the arm unit 11, for example. The user interface unit 570 outputs an operation signal corresponding to the received operation to the posture control unit 550. In this case, the posture control unit 550 controls the first joint 111₁ to the fourth joint 111₄ according to the operation received from the user interface unit 570 to control the position and the posture of the arm unit 11.

In the robot arm device 10, the electronic degree of freedom of changing the gaze line by cutting out the camera image captured by the camera 520 and the degree of freedom obtained by the actuator of the arm unit 11 are all treated as the degrees of freedom of the robot. This makes it possible to achieve execution of motion control that links the electronic degree of freedom of changing the gaze line and the degree of freedom obtained by the actuator.

<1-2-3. Processing Example of Medical Observation System>

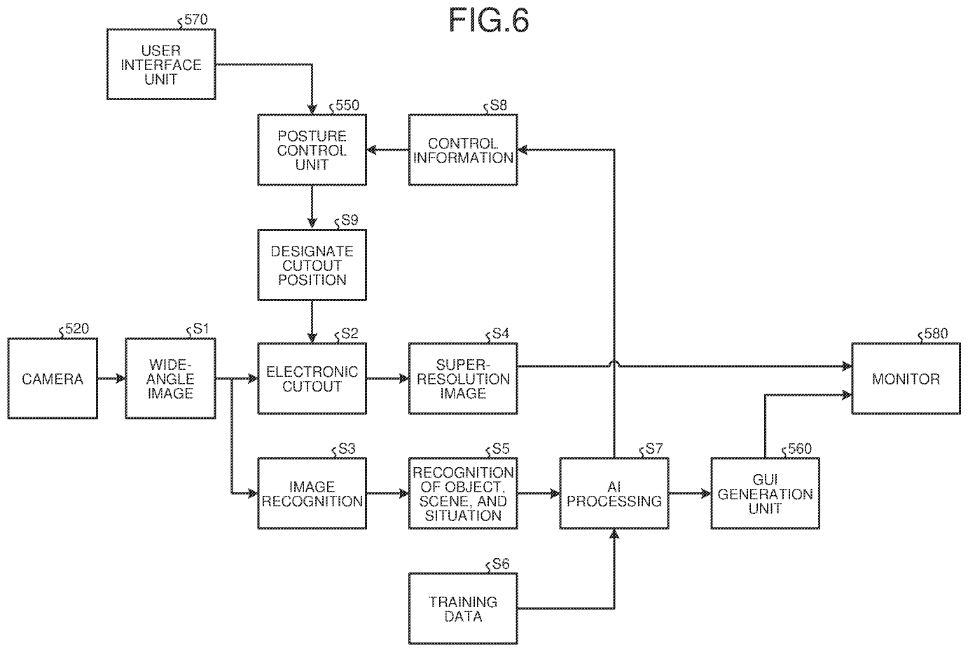

An example of a flow of processing of the medical observation system 1 according to the present embodiment will be described with reference to FIG. 6. FIG. 6 is a diagram illustrating an example of a flow of processing of the medical observation system 1 according to the present embodiment. As described above, the medical observation system 1 executes processing of combining the image recognition processing and the processing of controlling the position and posture of the robot arm device 10.

As illustrated in FIG. 6, first, in the medical observation system 1, the wide-angle image of the image capture target is captured by the camera 520 (step S1). On the basis of the wide-angle image captured by the camera 520, electronic cutout processing (step S2) for cutting out a video (for example, a cutout image) to be visually recognized by a doctor or the like and image recognition processing (step S3) for recognizing an operative field are executed. The processing of step S2 and the processing of step S3 may be executed in parallel.

It is also allowable to execute super-resolution processing on the video electronically cut out in step S2 to generate a super-resolution image (for example, a super-resolution cutout image) so as to allow the doctor to have better visual recognition of the video (step S4). The generated image is displayed on the monitor 580.

When the image recognition processing is executed in step S3, recognition results such as various objects, scenes, situations, and the like included in the image are output (step S5). The information regarding the recognition result is used at execution of artificial intelligence (AI) processing.

In order to autonomously control the position and posture of the camera 520, data related to a surgery being executed is input to a trained model (AI) that has learned in advance various types of data related to the surgery as training data (step S6). The various types data related to the surgery includes data such as an endoscopic image, information related to steering data of the endoscope by a doctor, operation information of the robot arm device 10, information (position/posture information) related to the position and posture of the arm unit 11, for example.

On the basis of the information related to various recognition results recognized in step S5 and the data related to the surgery input in step S6, AI processing for autonomously controlling the position and posture of the camera 520 is executed (step S7). As a result of the AI processing, control information for autonomously controlling the position of the camera 520 is output (step S8). In addition, the wide-angle image used in the image recognition processing in step S3 is input to the GUI generation unit 560. This allows the GUI generation unit 560 to display the wide-angle image of the operative field.

The control information output in step S8 is input to the posture control unit 550. The posture control unit 550 controls the position and posture of the camera 520. The position and posture of the camera 520 may be designated by the user interface unit 570.

The cutout position with respect to the wide-angle image is determined on the basis of the position and posture controlled by the posture control unit 550. Subsequently, the cutout position is designated on the basis of the determined cutout position (step S9). With this operation, the wide-angle image captured by the camera 520 is cut out again.

In the present embodiment, the processing illustrated in FIG. 6 is repeated to execute processing combining the image recognition processing and the processing of controlling the position and posture of the robot arm device 10.

<1-2-4. Example of Generation Processing of Wide-Angle Image and Cutout Image>

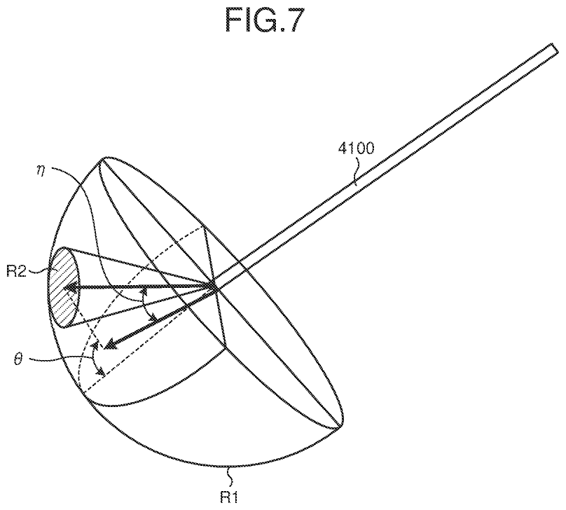

An example of processing of generating a wide-angle image and a cutout image according to the present embodiment will be described with reference to FIG. 7. FIG. 7 is a diagram illustrating an example of generation of a wide-angle image and a cutout image according to the present embodiment.

As illustrated in FIG. 7, an endoscope 4100 can image a wide-angle visual field R1 of a hemisphere (2π steradian). The endoscope 4100 corresponds to the endoscope 5001 and the imaging unit 12 described above. The image processing unit 21 generates a wide-angle image (first operative field image) corresponding to the wide-angle visual field R1, and further cuts out a display target region R2 desired by the user in the wide-angle visual field R1 to generate a cutout image (second operative field image). For example, the image processing unit 21 generates a cutout image by flexibly setting a pitch angle θ, a roll angle 11, and a viewing angle. The image processing unit 21 generates a cutout image by zooming in or out on the display target region R2.

Specifically, the image processing unit 21 generates a cutout image related to the display target region R2 which is a Region of Interest (ROI) that attracts the interest of the doctor in the wide-angle image. For example, the image processing unit 21 cuts out the display target region R2 in the wide-angle image, thereby generating a cutout image related to the display target region R2. As an example, the image processing unit 21 generates a cutout image by cutting out and enlarging the display target region R2 in the wide-angle image. In this case, the image processing unit 21 may change the cutout position according to the position and posture of the arm unit 11. For example, the image processing unit 21 changes the cutout position so that the cutout image displayed on the display screen does not change when the position and posture of the arm unit 11 have been changed. The display target region R2 may be designated, for example, by a user such as a doctor or an assistant using the input device 5047 as an operation unit (user designation), or may be judged on the basis of a recognition result obtained by the image processing unit 21.

Conventionally, three degrees of freedom of pitch, roll, and zoom in a straight-viewing endoscope and four degrees of freedom of pitch, roll, zoom, and yaw in an oblique-viewing endoscope have been achieved by changing the position and posture of the straight-viewing endoscope or the oblique-viewing endoscope by using a mechanical degree of freedom outside the patient's body. In contrast, the present embodiment uses the configuration as illustrated in FIG. 7, and thus, a system electronically having three degrees of freedom of a pitch, a roll, and a zoom can achieve the movement equivalent to the movement conventionally desired without accompanying movement of a mechanism outside the body. In addition, it is also possible to achieve an operation that has been restricted in movement with conventional endoscope, such as achievement of a look-around motion with a constant distance to a target.

For example, in order to achieve the look-around motion while continuously capturing one point of the observation target with a conventional technology, it has been necessary to move the endoscope in a conical motion with an observation axis of the endoscope facing the point. In contrast, the present embodiment makes it possible to freely take the posture of the look-around motion with a constant distance to the target in the wide-angle visual field R1 without a need to move the endoscope 4100 (for example, an oblique-viewing endoscope) in a conical shape in that manner. In addition, regarding the movement of changing the direction of looking around while zooming the endoscope in the observation axis direction, it is possible to look around while keeping a constant magnification rate of the target by adding an electronic zoom operation. Furthermore, it is possible, in the present embodiment, to electronically execute operations of the pitch and roll of the endoscope, leading to achievement of prevention of interference between the operation of the pitch and roll of the endoscope and the operation performed by the doctor. This improves operability of the doctor. In addition, by electronically executing the operation of the pitch and roll of the endoscope, it is possible to eliminate the operation of manually moving the endoscope by the doctor when looking around the observation target. This improves operability of the doctor.

<1-2-5. Detailed Configuration Example of Gaze Processing Unit>

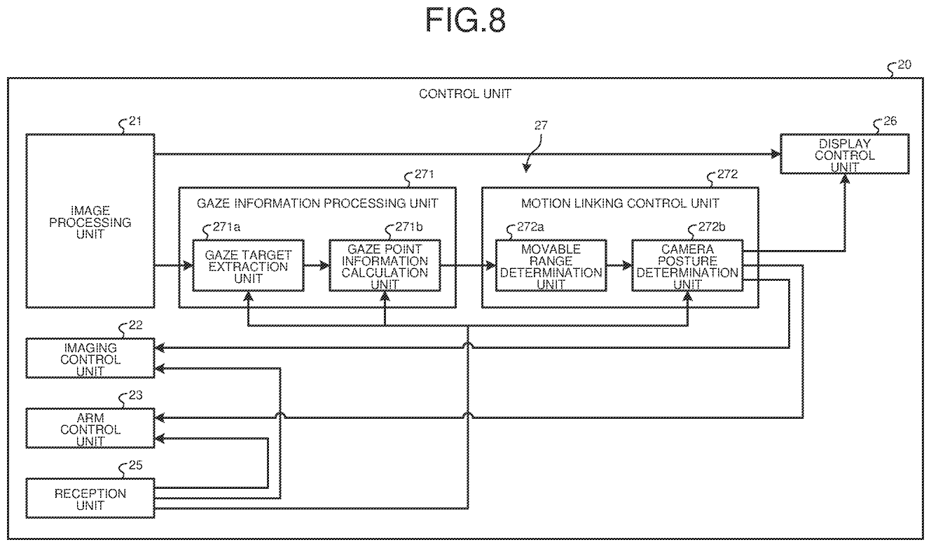

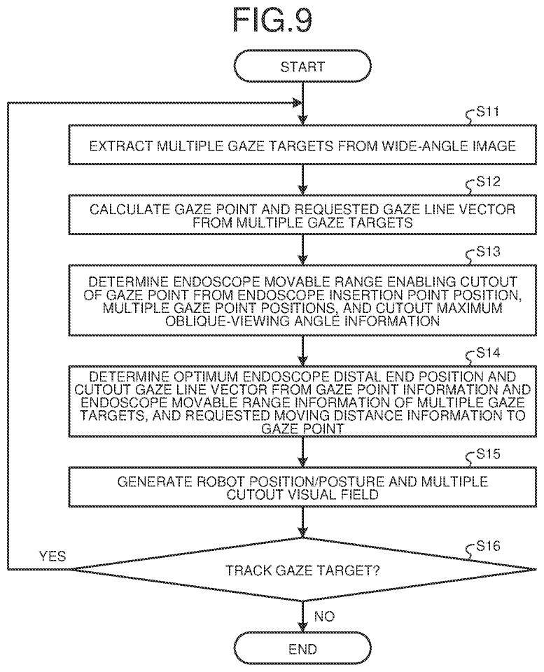

An example of a detailed configuration of the gaze processing unit 27 according to the present embodiment will be described with reference to FIGS. 8 and 9. FIG. 8 is a diagram illustrating an example of a detailed configuration of the gaze processing unit 27 according to the present embodiment. FIG. 9 is a flowchart illustrating an example of basic processing according to the present embodiment.

As illustrated in FIG. 8, the gaze processing unit 27 includes a gaze information processing unit 271 and a motion linking control unit 272. The gaze information processing unit 271 includes a gaze target extraction unit 271a and a gaze point information calculation unit 271b. The motion linking control unit 272 includes a movable range determination unit 272a and a camera posture determination unit (posture determination unit) 272b. These units will be described along the flow of processing.

As illustrated in FIG. 9, the gaze target extraction unit 271a extracts, in step S11, multiple gaze targets from a wide-angle image. In step S12, the gaze point information calculation unit 271b calculates a gaze point and a requested gaze line vector from multiple gaze targets. In step S13, the movable range determination unit 272a determines the endoscope movable range enabling the cutout of the gaze point from an endoscope insertion point position (distal end position of the endoscope 4100), the multiple gaze point positions, and the cutout maximum oblique-viewing angle information.

In step S14, the camera posture determination unit 272b determines an optimum endoscope distal end position and a cutout gaze line vector from the gaze point information of the multiple gaze targets, the endoscope movable range information, and the requested moving distance information to the gaze point. In step S15, the camera posture determination unit 272b generates robot position/posture and a multiple cutout visual field from the optimum endoscope distal end position and cutout gaze line vector. The robot position/posture and the multiple cutout visual field are generated as posture information (part of control information).

In step S16, the gaze processing unit 27 judges whether to continue gaze target tracking. When it is judged to continue gaze target tracking (Yes), the gaze processing unit 27 returns the processing to step S11. In contrast, when it is judged that the gaze target tracking is not continued (No), the processing ends.

Although multiple gaze targets is extracted in step S11, the number of extracted gaze targets is not particularly limited, and a single gaze target may be extracted. Similarly to the above, steps S11 to S16 are also executed for the single gaze target.

<1-2-6. Detailed Processing Example of Gaze Processing Unit>

An example of detailed processing of the gaze processing unit 27 according to the present embodiment will be described along the flow of processing (a to e).

(a. Extraction of Gaze Target from Wide-Angle Image)

First, the imaging unit 12 acquires a wide-angle image (first operative field image) from the endoscope 4100. The imaging unit 12 functions as an image input unit. Note that the image processing unit 21 may perform image processing such as distortion correction as necessary. The wide-angle image after this processing is used as an image to be input to subsequent image recognition processing and the like. Here, image recognition processing is used on the processed wide-angle image, and gaze target extraction and subsequent image cutout processing are performed.

(b. Calculation of Gaze Point Information)

Next, the gaze target extraction unit 271a calculates gaze point information related to a gaze point of the gaze target. The gaze point information includes position information of a gaze point of the gaze target and vector information of a requested gaze line vector, for example.

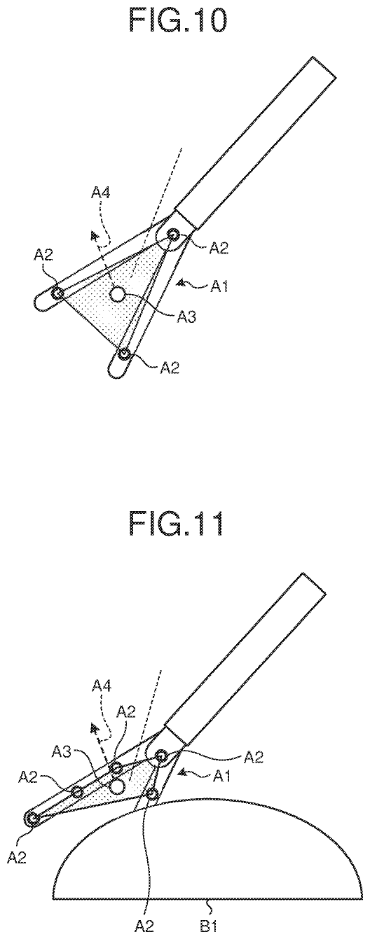

FIG. 10 is a diagram illustrating gaze point information calculation according to the present embodiment. As illustrated in FIG. 10, the gaze target A1 includes multiple feature points A2 (feature point clouds). For example, each feature point A2 is detected and set by a recognition technology such as instrument recognition or organ recognition, or is set by user designation represented by input information received by the reception unit 25, but the setting method is not limited. Note that recognition processing such as instrument recognition and organ recognition is executed on the basis of data (for example, a learning model or the like) input to the image recognition engine in advance.

The gaze point information calculation unit 271b detects the gaze target A1 and obtains each feature point A2. Next, the gaze point information calculation unit 271b calculates gaze point A3 and requested gaze line vector A4. At this time, for example, the gaze point information calculation unit 271b calculates a “center of gravity” based on the three-dimensional position information of each feature point A2, and calculates a “gaze target plane” to be fitted to the feature point cloud using the least squares method or the like. The three-dimensional position information of each feature point A2 is calculated using position information on the camera image, depth information, and the like based on image recognition. Next, the gaze point information calculation unit 271b calculates an intersection of perpendicular lines drawn from the center of gravity onto the gaze target plane as a “gaze point A3” and a normal vector from the gaze target plane toward the center of gravity as a “requested gaze line vector A4”, and uses the calculation result to obtain the position and posture of the endoscope 4100 and the cutout gaze line vector. The position information of the “gaze point A3” and the vector information regarding the “requested gaze line vector A4” are associated with each other and are treated as “gaze point information”.

Note that it is also allowable to add a process of determining whether or not to adopt the calculated “gaze point information” after evaluation by the user. This makes it possible to eliminate the requested gaze line vector not intended by the user, enabling an endoscope movement and presentation of the cutout image closer to the user request. In addition to the recognition processing, the feature point A2 and the gaze point A3 may be set on the basis of input information received by the reception unit 25 (for example, input information designated by the user), for example.

Here, FIG. 11 is a diagram illustrating an example in which the feature point A2 is missing due to an obstacle B1. As illustrated in FIG. 11, there is a case where a part of the gaze target A1 cannot be visually recognized due to the obstacle B1. Even in this case, the gaze target A1 includes multiple feature points A2 (feature point cloud). With this configuration, even in a case where a part of the gaze target A1 cannot be visually recognized by the obstacle B1, the gaze point information can be calculated. That is, the gaze point information can be calculated even in a state where a part of each feature point A2 is not captured by the endoscope 4100.

(c. Determination of Endoscope Movable Range)

Subsequently, the movable range determination unit 272a determines a movable range of the endoscope distal end position (endoscope movable range) for achieving cutout of the gaze point A3 (generation of a cutout image including the gaze point A3).

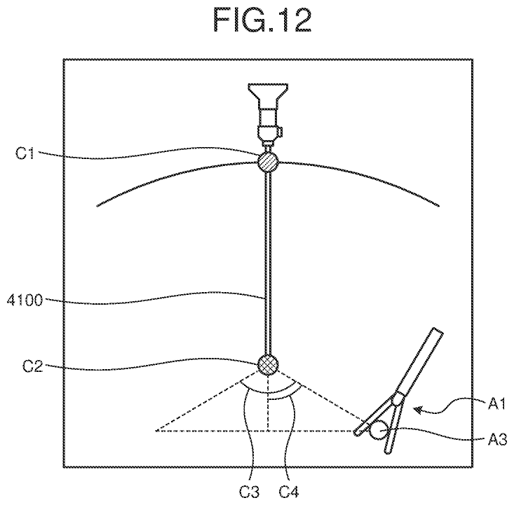

FIG. 12 is a diagram illustrating a cutout maximum oblique-viewing angle according to the present embodiment. In the example of FIG. 12, an insertion point (endoscope insertion point) of the endoscope 4100 is C1, and a distal end point (endoscope distal end point) of the endoscope 4100 is C2. As illustrated in FIG. 12, the endoscope 4100 has viewing angle information related to a viewing angle C3 determined in advance as a specification. Therefore, the “cutout maximum oblique-viewing angle (maximum oblique-viewing angle C4)” in the case of performing image cutout display corresponding to the oblique-viewing endoscope by a screen cutout function is determined from the viewing angle information.

(c-1. Determination of Endoscope Movable Range at Single Gaze Point)

The movable range determination unit 272a uses the calculated “gaze point information”, the position information of the “endoscope insertion point”, and the information of the “cutout maximum oblique-viewing angle” calculated from the viewing angle of the wide-angle endoscope to determine the “endoscope movable range” enabling cutout of the gaze point.

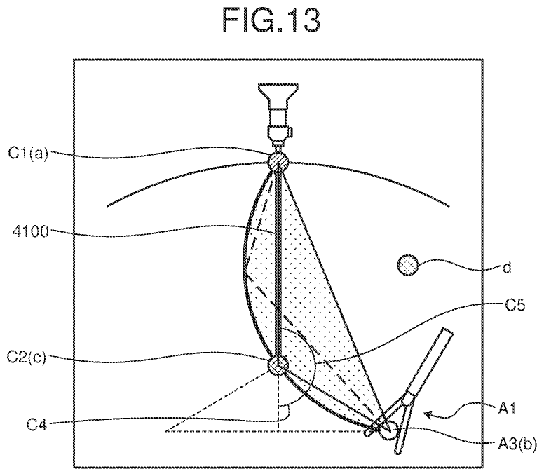

FIG. 13 is a diagram illustrating endoscope movable range determination of a single gaze point according to the present embodiment. In the example of FIG. 13, there is one gaze target A1. Therefore, as illustrated in FIG. 13, one gaze point A3 is detected. When the endoscope insertion point C1 is a, the gaze point A3 is b, and the endoscope distal end point C2 is c, the point c on a circumscribed circle of a triangle abc, specifically a circumscribed circle having a circumcenter d at which the circumferential angle C5 of an arc ab is (180°-cutout maximum oblique-viewing angle), is calculated as the endoscope distal end position enabling cutout of the gaze point A3 at the maximum oblique-viewing angle C4. At this time, the “endoscope movable range” enabling cutout of the gaze point A3 (an image including gaze point A3) is defined by a region including a line ab and the arc ab passing through the point c (region filled with dots in FIG. 13). The “endoscope movable range” indicates a movement range of the endoscope distal end position enabling cutout display of the gaze point A3 between the minimum oblique-viewing angle (straight viewing) and the maximum oblique-viewing angle C4. An endoscope movable range in a three-dimensional space in reality is a region obtained by expanding the endoscope movable range on a spherical surface.

(c-2. Determination of Endoscope Movable Range for Multiple Gaze Points)

The “endoscope movable range” enabling simultaneous cutout of the multiple gaze points A3 is defined by a common portion being an overlapping portion of the “endoscope movable ranges” calculated at the single gaze point A3, and is referred to as a “multiple gaze point cutout endoscope movable range”.

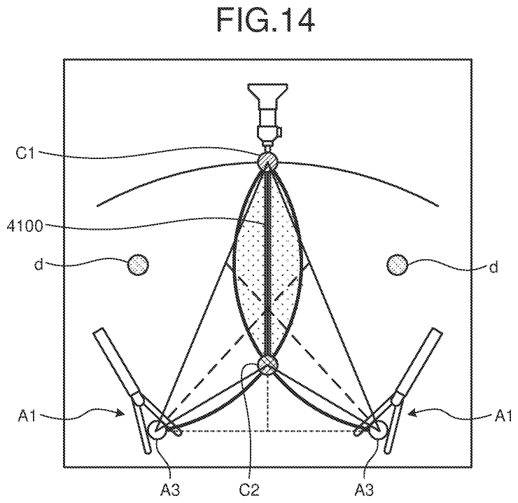

FIG. 14 is a diagram illustrating determination of an endoscope movable range of the multiple gaze points A3 according to the present embodiment. In the example of FIG. 14, there are two gaze targets A1. Therefore, as illustrated in FIG. 14, two gaze points A3 (first gaze point A3 and second gaze point A3) are detected. The “multiple gaze point cutout endoscope movable range” is defined as a range enabling simultaneous cutout of all the gaze points A3. The “multiple gaze point cutout endoscope movable range” is a region where the movable ranges of individual gaze points A3 overlap each other (a region filled with dots in FIG. 14).

However, depending on the position and the number of gaze points A3, there may be a case where the “multiple gaze point cutout endoscope movable range” does not exist. In this case, the camera posture determination unit 272b determines the position/posture and the cutout gaze line vector of the endoscope 4100 on the basis of the requested level (priority information) of the gaze point A3 by using both the “endoscope movable range” information calculated at the individual gaze points A3 and the “multiple gaze point cutout endoscope movable range” information calculated from the multiple gaze points A3 (details will be described below). Note that the requested level of the gaze point A3 may be set on the basis of, for example, input information (for example, input information designated by the user) received by the reception unit 25, or may be set according to a use case or information such as a type of an instrument or an organ.

(d. Determination of Endoscope Position and Cutout Gaze Line Vector)

The camera posture determination unit 272b determines the position (distal end position) and posture of the endoscope 4100 and the cutout gaze line vector from the information including the “gaze point information” and the “endoscope movable range”.

(d-1. Determination of Endoscope Position and Cutout Gaze Line Vector for Single Gaze Point)

In the use case for a single gaze point, the camera posture determination unit 272b determines the endoscope position and the cutout gaze line vector by using the gaze point position and the requested gaze line vector information.

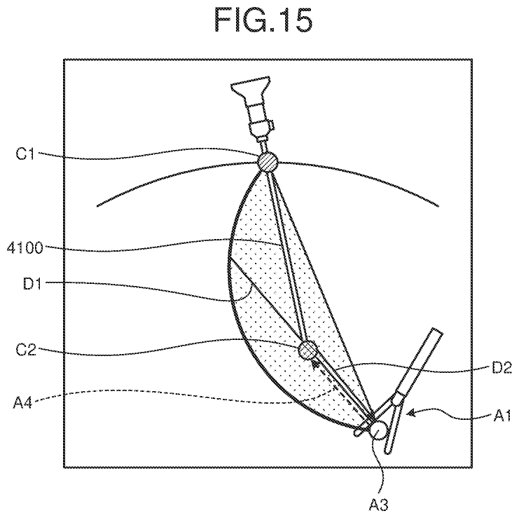

FIG. 15 is a diagram illustrating an endoscope distal end requested movement trajectory in a case where the requested gaze line vector A4 according to the present embodiment is within the endoscope movable range. As illustrated in FIG. 15, in a case where a straight line D1 on the extension of the requested gaze line vector A4 passes through the endoscope movable range, a point cloud on the straight line within the movable range is position information indicating the position where the distal end of the endoscope 4100 should move. This position information is referred to as an “endoscope distal end requested movement trajectory”. A cutout gaze line vector D2 is a vector in a direction opposite to the requested gaze line vector A4.

Here, the vector information need not be used when the gaze target A1 is moving. For example, the vector information need not be used when the gaze point A3 is moving. When the gaze point A3 is stopped, it is allowable to use vector information related to the gaze point A3 in the stopped state. In this case, tracking may be performed on only the gaze point A3 in the stopped state. In addition, the tracking performance in tracking the gaze point A may be reduced in accordance with an increase in the moving speed of the gaze point A3, for example, gradually or in a case where the moving speed exceeds a threshold.

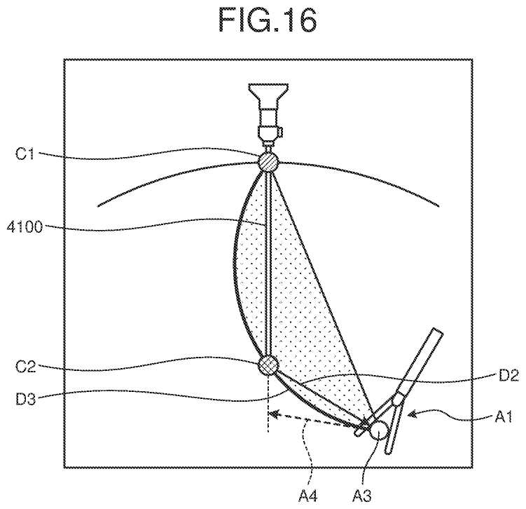

FIG. 16 is a diagram illustrating an endoscope distal end requested movement trajectory in a case where the requested gaze line vector A4 according to the present embodiment is outside the endoscope movable range. As illustrated in FIG. 16, in a case where the straight line D1 on the extension line of the requested gaze line vector A4 does not pass through the endoscope movable range, a trajectory (point cloud on the circumscribed circle) D3 enabling the maximum oblique-viewing angle on the plane closest to the requested gaze line vector A4 within the endoscope movable range is set as the “endoscope distal end requested movement trajectory”. At this time, the cutout gaze line vector D2 becomes a vector from the endoscope distal end point C2 toward the gaze point A3. The final position on the “endoscope distal end requested movement trajectory” is determined on the basis of a requested distance to the gaze point A3 and the like. Note that the requested distance may be set on the basis of, for example, input information (for example, input information designated by the user) received by the reception unit 25, or may be set according to a use case or information such as a type of an instrument or an organ.

(d-2. Determination of Endoscope Positions and Cutout Gaze Line Vectors for Multiple Gaze Points)

In a use case for multiple gaze points, the camera posture determination unit 272b gives priority to a requested gaze line vector of a specific gaze point. Specifically, similarly to the single gaze point, the camera posture determination unit 272b determines the endoscope distal end position from the “gaze point information” and the “endoscope movable range” information of each gaze point. For example, the camera posture determination unit determines the endoscope position and the cutout gaze line vector by using the requested gaze line vector information of a specific gaze point having the highest priority.

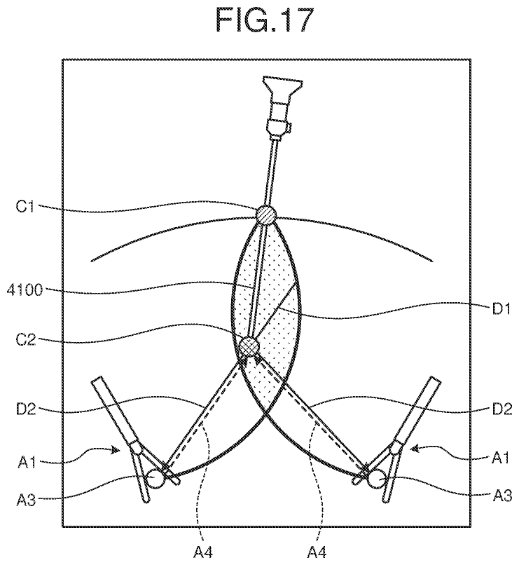

FIG. 17 is a diagram illustrating an endoscope distal end requested movement trajectory in a case where a requested gaze line vector of each gaze point A3 is within an endoscope movable range according to the present embodiment. As illustrated in FIG. 17, in a case where the straight line D1 on the extension of the requested gaze line vector A4 of a specific gaze point A3 (In the example of FIG. 17, the left gaze point A3) passes through the endoscope movable range, the point cloud on the straight line within the movable range becomes the “endoscope distal end requested movement trajectory”, and the cutout gaze line vector D2 of the specific gaze point A3 becomes a reverse vector of the requested gaze line vector A4 of the specific gaze point A3. In addition, the cutout gaze line vector D2 of each gaze point A3 (in the example of FIG. 17, gaze point A3 on the right side) other than the specific gaze point A3 is a vector from the endoscope distal end position determined above toward each gaze point A3.

Similarly to the case of the single gaze point, the final position on the “endoscope distal end requested movement trajectory” may be determined on the basis of the requested distance to the gaze point A3. As another method, the final position may be determined on the basis of the requested gaze line vector information of another gaze point A3. In this case, the camera posture determination unit 272b determines, as the endoscope distal end position, a point on the “endoscope distal end requested movement trajectory” that minimizes a difference (an angle formed between vectors) between the cutout gaze line vector D2 of each of the gaze points A3 other than the specific gaze point A3 and the requested gaze line vector A4 of each of the gaze points A3.

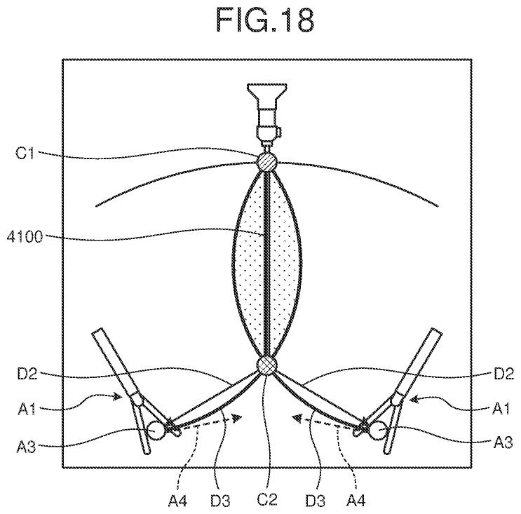

FIG. 18 is a diagram illustrating an endoscope distal end requested movement trajectory in a case where a requested gaze line vector A4 of each gaze point A3 is outside an endoscope movable range according to the present embodiment. As illustrated in FIG. 18, also in a case where the straight line D1 (refer to FIG. 17) on the extension of the requested gaze line vector A4 does not pass through the endoscope movable range, the camera posture determination unit 272b performs, similarly to the above description, setting of the trajectory (point cloud on the circumscribed circle) D3 enabling the maximum oblique-viewing angle similarly to the case of the single gaze point as the “endoscope distal end requested movement trajectory” from the gaze point information of the specific gaze point A3, and determination related to the endoscope distal end position, that is, determination of the optimum endoscope distal end position similarly to the case of passing through the movable range.

(d-3. Determination of Endoscope Positions and Cutout Gaze Line Vectors for Multiple Gaze Points)

In the use case that uses an average requested gaze line vector, in order to perform on-average capture and tracking of the all gaze points A3 on the screen, the camera posture determination unit 272b uses all the requested gaze line vectors A4 of the multiple gaze points A3 to calculate the average requested gaze line vector to perform the tracking.

According to this system, two vectors are selected from multiple three-dimensional requested gaze line vectors A4, and calculates an average requested gaze line vector of the two vectors under a straight line condition, that is, a condition of passing through a common perpendicular of two straight lines on an extension of the two vectors and being parallel to the two straight lines. By repeating this processing on all the gaze points A3 of subsequent priorities, the average requested gaze line vectors of all the requested gaze line vectors are calculated. By adopting an inverse vector of the average requested gaze line vector as the cutout gaze line vector D2 of the endoscope 4100, it is possible to capture all gaze points A3 in a direction satisfying the requested gaze lines of the all gaze points A3 on average.

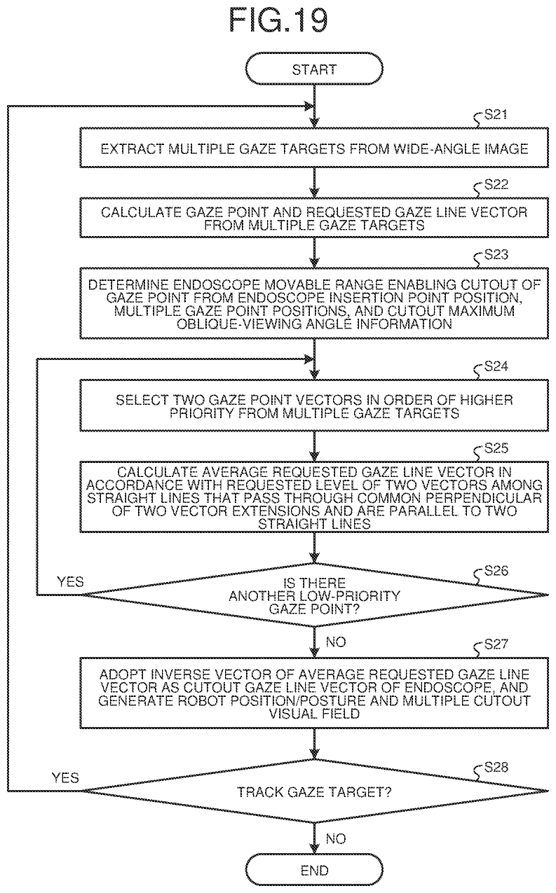

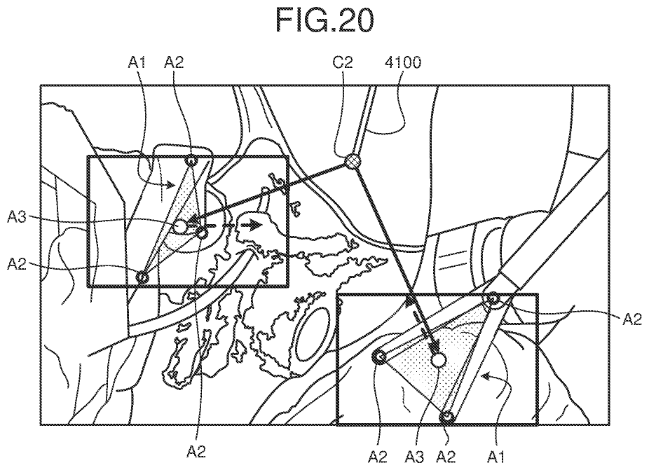

FIG. 19 is a flowchart illustrating a flow of processing of calculating the average requested gaze line vector of all the gaze points A3 and performing tracking according to the present embodiment. As illustrated in FIG. 19, the gaze target extraction unit 271a extracts, in step S21, multiple gaze targets from a wide-angle image. In step S22, the gaze point information calculation unit 271b calculates a gaze point and a requested gaze line vector from multiple gaze targets. In step S23, the movable range determination unit 272a determines the endoscope movable range enabling cutout of the gaze point, from an endoscope insertion point position, the multiple gaze point positions, and the cutout maximum oblique-viewing angle information.

In step S24, the camera posture determination unit 272b selects two gaze point vectors in order of higher priority from the multiple gaze targets. In step S25, the camera posture determination unit 272b calculates an average requested gaze line vector in accordance with the requested level of two vectors among straight lines that pass through the common perpendicular of the two vector extensions and are parallel to the two straight lines. In step S26, the camera posture determination unit 272b judges whether or not there is another low-priority gaze point. When judged that there is another low-priority gaze point (Yes), the processing returns to step S21. In contrast, when it is judged that there is no other low-priority gaze point (No), the processing proceeds to step S27.

In step S27, the camera posture determination unit 272b adopts an inverse vector of the average requested gaze line vector as the cutout gaze line vector of the endoscope 4100, and generates the robot position/posture and the multiple cutout visual field. The robot position/posture and the multiple cutout visual field (cutout gaze line vectors) are generated as control information. In step S28, the gaze processing unit 27 judges whether to continue gaze target tracking. When it is judged to continue gaze target tracking (Yes), the processing returns to step S21. In contrast, when it is judged that the gaze target tracking is not continued (No), the processing ends.

(e. Endoscope Position Operation and Screen Cutout Operation)

The arm control unit 23 controls the robot arm device 10 on the basis of the calculated position and posture of the distal end of the endoscope to automatically operate the endoscope 4100.

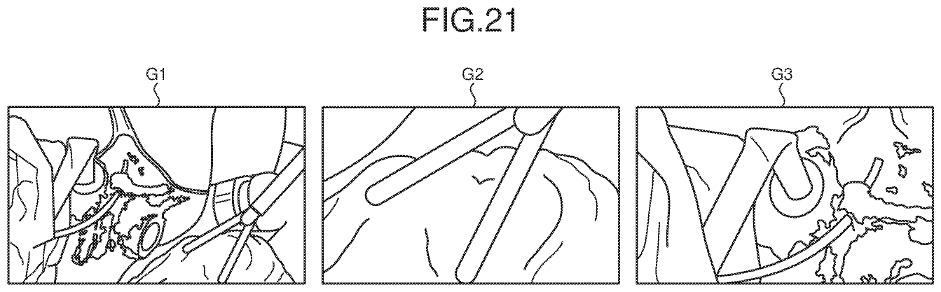

FIG. 20 is a diagram illustrating an endoscope distal end position and a cutout gaze line vector D2 at the time of multiple gaze point cutout according to the present embodiment. In the example of FIG. 20, there are two gaze targets A1. Therefore, as illustrated in FIG. 20, two gaze points A3 (first gaze point A3 and second gaze point A3) are detected. For example, together with the change of the endoscope position by the arm control, the image processing unit 21 cuts out and generates a cutout image for multiple gaze points A3 from the wide-angle image on the basis of multiple cutout gaze line vector information, and outputs individual cutout images (the first gaze point cutout image and the second gaze point cutout image) to the presentation device 40. The image processing unit 21 functions as a cutout image generation unit.

FIG. 21 is a diagram illustrating an example of an image at the time of multiple gaze point cutout according to the present embodiment. In each image illustrated in FIG. 21, a left image G1 is a wide-angle image, a center image G2 is a first gaze point cutout image, and a right image G3 is a second gaze point cutout image. As illustrated in FIG. 21, for example, the presentation device 40 displays the cutout image and the wide-angle image for each gaze point A3 on the same screen so as not to overlap each other. This makes it possible for the surgeon 5067 to perform a surgical operation while visually recognizing these images. Accordingly, the surgeon 5067 can grasp the states of the surgical site in more detail, leading to smooth progress of the surgery. Incidentally, multiple display devices may be provided as the presentation device 40, and each cutout image may be displayed on each display device in synchronization with displaying the wide-angle image on one display device.

<1-3. First Modification>

A first modification of the present embodiment is a use case of performing simply tracking of a gaze point. This use case is a simple tracking system that merely captures a gaze point within a screen without using a requested gaze line vector of a gaze point.

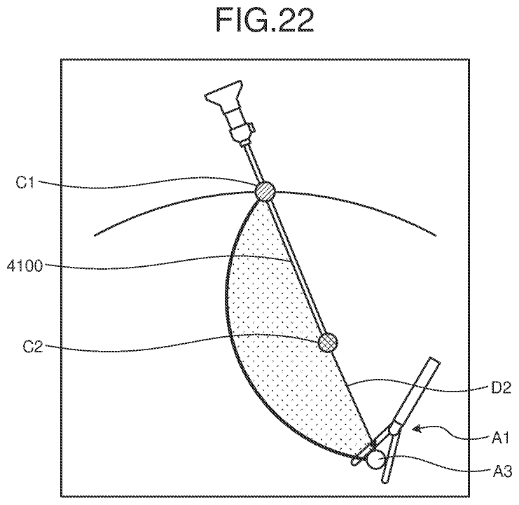

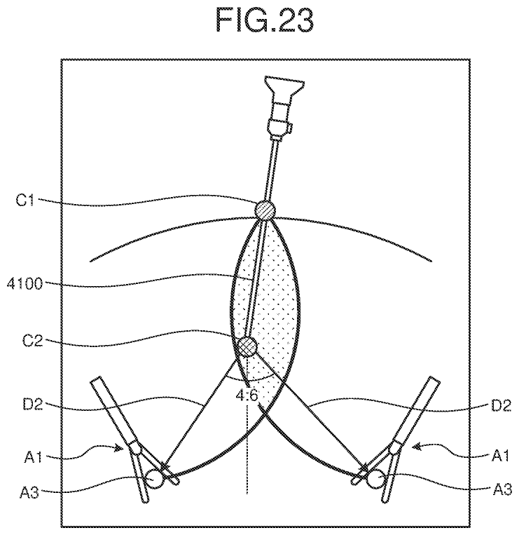

FIG. 22 is a diagram illustrating generation of a straight-viewing cutout gaze line vector of a single gaze point A3 according to the first modification of the present embodiment. As illustrated in FIG. 22, in the single gaze point use case, it is possible to calculate and control the position/posture of the endoscope 4100 for capturing the gazing point A3 in a mode close to straight viewing as the center without referring to the requested gaze line vector A4 (refer to FIG. 15).

FIG. 23 is a diagram illustrating distal end position determination according to a requested level (ratio) of multiple gaze points A3 according to the first modification of the present embodiment. As illustrated in FIG. 23, in the multiple gaze point use case, when not referring to the requested gaze line vector A4 (refer to FIG. 15), the endoscope distal end position can be calculated according to the requested level (for example, the ratio value) of each gaze point A3. In the example of FIG. 23, the ratio value is 4:6. Specifically, the gaze processing unit 27 simply weights the cutout gaze line vector D2 according to the requested level (for example, the ratio value) with respect to the endoscope standard position where the cutout gaze line vectors D2 to the two gaze points A3 have the same angle, enabling tracking and image cutout in a mode closer to straight viewing by the cutout gaze line vector D2 for the gaze point A3 with a high requested level.

For example, in an actual surgical use case, by changing the requested level of the gaze point A3 according to the scene, it is possible to switch the gaze target A1 to be captured in a mode close to straight viewing while maintaining the tracking and the image cutout display of all the gaze points A3. The requested level is a level indicating the priority of the cutout gaze line vector D2.

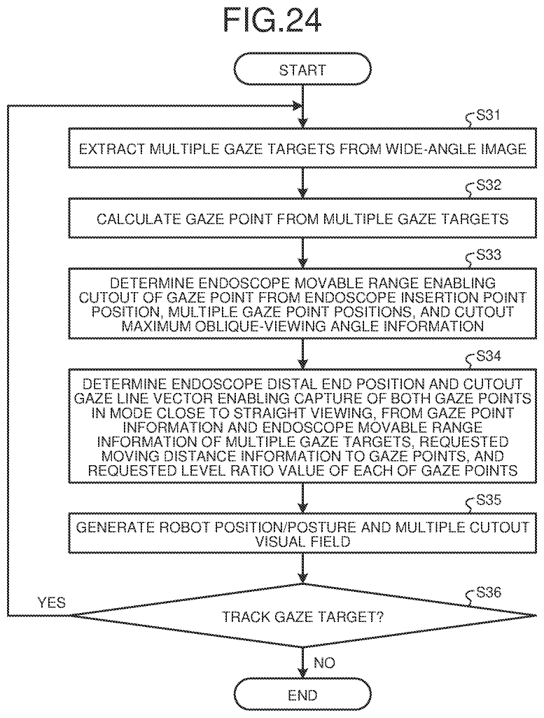

FIG. 24 is a flowchart illustrating a flow of processing of a requested gaze line vector non-reference case according to the first modification of the present embodiment. As illustrated in FIG. 24, the gaze target extraction unit 271a extracts, in step S31, multiple gaze targets from the wide-angle image. In step S32, the gaze point information calculation unit 271b calculates a gaze point from multiple gaze targets. In step S33, the movable range determination unit 272a determines the endoscope movable range enabling cutout of the gaze point from an endoscope insertion point position, the multiple gaze point positions, and the cutout maximum oblique-viewing angle information.

In step S34, from the gaze point information and the endoscope movable range information of the multiple gaze targets, the requested moving distance information to the gaze point, and the requested level ratio value of each gaze point, the camera posture determination unit 272b determines an optimal endoscope distal end position and a cutout gaze line vector, that is, an endoscope distal end position and a cutout gaze line vector enabling the capture of each gaze point in a mode close to straight viewing. In step S35, the camera posture determination unit 272b generates the robot position/posture and the multiple cutout visual field from the optimal endoscope distal end position and cutout gaze line vector. The robot position/posture and the multiple cutout visual field (cutout ranges) are generated as control information.

In step S36, the gaze processing unit 27 judges whether to continue gaze target tracking, and when it is judged to continue gaze target tracking (Yes), the processing returns to step S31. In contrast, when it is judged that the gaze target tracking is not continued (No), the processing ends.

Although multiple gaze targets is extracted in step S31, the number of extracted gaze targets is not particularly limited, and a single gaze target may be extracted. Similarly to the above, steps S31 to S36 are also executed for the single gaze target.

<1-4. Second Modification>

A second modification of the present embodiment is a virtual wall setting use case using endoscope movable range information. In this use case, endoscope movable range information enabling simultaneous cutout of a screen at multiple gaze points is used not only in an automatic tracking operation by an endoscope robot (for example, the robot arm device 10) but also as a virtual wall function that limits an operation region when a user performs manual operations.

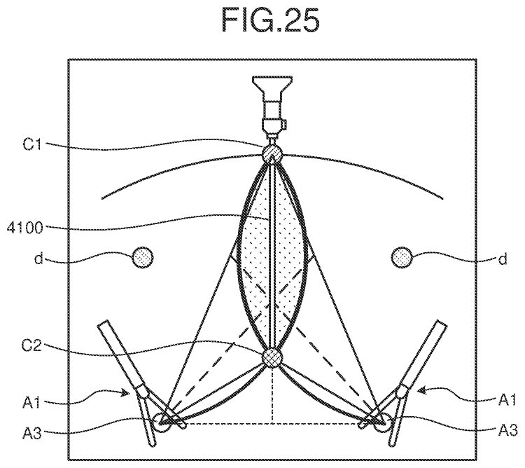

FIG. 25 is a diagram illustrating virtual wall setting by an endoscope movable range of multiple gaze points A3 according to the second modification of the present embodiment. As illustrated in FIG. 25, an overlapping range of the movable ranges of the respective gaze points A3 (region filled with dots in FIG. 25) is a cutout endoscope movable range of the multiple gaze points A3. The distal end of the endoscope 4100 is restricted from protruding from this movable range, leading to an operation of the position and posture of the endoscope 4100. That is, a boundary between the movable region and a region (a region other than the movable region) that restricts the position and posture of the endoscope 4100 functions as a virtual wall. With this function, even at the time of manual operation by the user, it is possible to perform an endoscope position/posture operation while maintaining a state where multiple gaze points A3 is captured as a cutout image.

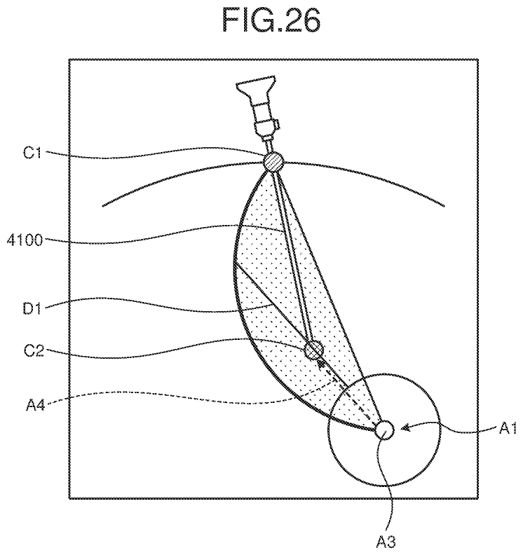

FIG. 26 is a diagram illustrating a contact avoidance operation by endoscope approach prohibition distance setting according to the second modification of the present embodiment. As illustrated in FIG. 26, by adding an approach prohibition distance constraint as the virtual wall to be the endoscope movement region restriction at the time of calculating the “endoscope distal end requested movement trajectory” for the gaze point A3 having an approach risk such as an organ, it is possible to achieve operations of the gaze point tracking operation and the gaze point contact avoidance operation while presenting a gaze point cutout image. That is, a virtual wall is added on the basis of an approach prohibition region (a perfect circle region around the gaze point A3 in FIG. 26), which is a region prohibiting the endoscope 4100 from approaching the gaze point A3. Note that the approach prohibition region (as an example, the approach prohibition distance) may be set on the basis of, for example, input information received by the reception unit 25 (as an example, input information designated by the user), or may be set according to a use case or information such as the type of an instrument or an organ.

For example, in an actual surgical use case, specifically in a scene where the treatment of the specific gaze point A3 is performed, the procedure is performed using a specific gaze point A3 as a main reference image with an organ or the like having a risk of damage being recognized as another gaze point A3 having a contact avoidance request, making it possible to achieve the contact avoidance operation.

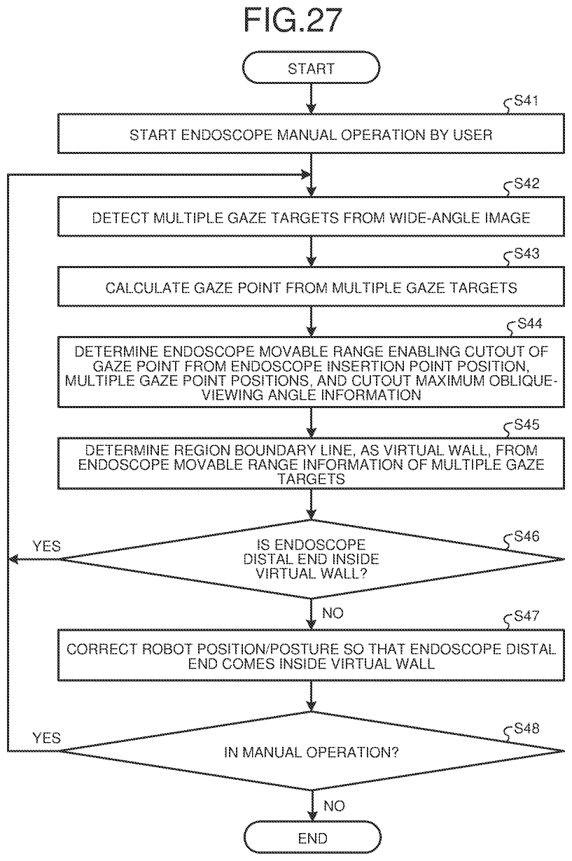

FIG. 27 is a flowchart illustrating a flow of processing of a virtual wall setting case based on endoscope movable range information according to the second modification of the present embodiment. As illustrated in FIG. 27, an endoscope manual operation by the user is started in step S41. In step S42, the gaze target extraction unit 271a extracts multiple gaze targets from the wide-angle image. In step S43, the gaze point information calculation unit 271b calculates a gaze point from multiple gaze targets.

In step S44, the movable range determination unit 272a determines the endoscope movable range enabling cutout of the gaze point, from an endoscope insertion point position, the multiple gaze point positions, and the cutout maximum oblique-viewing angle information. In step S45, the movable range determination unit 272a sets a region boundary line as a virtual wall from the endoscope movable range information of the multiple gaze targets. In step S46, the camera posture determination unit 272b judges whether or not the endoscope distal end is inside the virtual wall. When it is judged that the endoscope distal end is inside the virtual wall (Yes), the processing returns to step S42. In contrast, when it is judged that the endoscope distal end is not inside the virtual wall (No), the processing proceeds to step S47.

In step S47, the camera posture determination unit 272b corrects the robot position/posture such that the distal end of the endoscope comes inside the virtual wall. In step S48, it is judged whether or not the arm operation is in a manual operation, and when it is judged that the arm operation is in the manual operation (Yes), the processing returns to step S42. In contrast, when it is judged that the arm operation is not in the manual operation (No), the processing ends.

Note that, in the second modification, it has been exemplified that the virtual wall is set, but the present invention is not limited thereto. For example, a warning image indicating that the distal end of the endoscope 4100 exceeds the endoscope movable range may be presented by the presentation device 40 without setting the virtual wall. Furthermore, even in a case where the virtual wall is set, the warning image described above may be presented by the presentation device 40 in addition to the correction of the robot position/posture described above. As the warning image, in addition to the warning image indicating that the distal end of the endoscope 4100 exceeds the endoscope movable range, it is also allowable to use a warning image indicating that the distal end of the endoscope 4100 is about to exceed the endoscope movable range (for example, an image indicating that the distal end exceeds a position of a predetermined distance inward from the boundary of the endoscope movable range).

<1-5. Third Modification>

A third modification of the present embodiment is a use case of tracking visual field movement from a single gaze point to a different gaze point. In this use case, in a case where the endoscope 4100 moves from a first movable range to a second movable range according to the visual field movement from the first gaze point to the second gaze point, the robot arm device 10 is controlled to minimize the moving distance of the endoscope 4100.

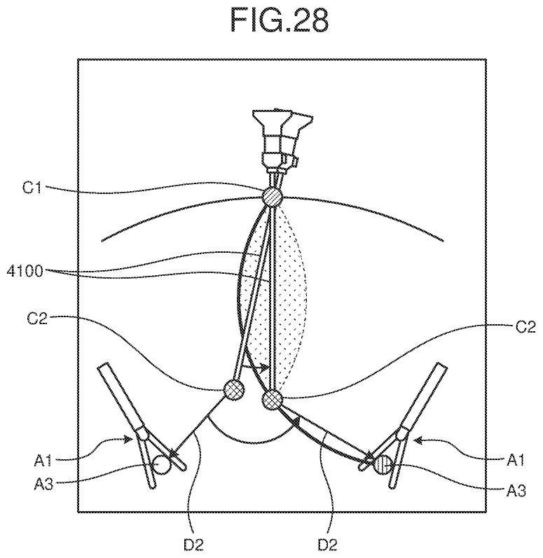

FIG. 28 is a diagram illustrating minimization of an endoscope posture change amount at the time of cutout visual field movement according to a third modification of the present embodiment. As illustrated in FIG. 28, in the cutout display use case of the single gaze point A3, in a case where the visual field is moved to a different gaze point A3 (for example, from the first gaze point A3 to the second gaze point A3), a vector minimizing the distance of the endoscope movable range region calculated from a movement source gaze point A3 and a movement destination gaze point A3 is calculated and adopted as a movement vector of the endoscope 4100. This makes it possible to perform a display target switching operation while minimizing the posture change of the endoscope 4100 in the cutout display target switching operation accompanying an endoscope posture change.

For example, in an actual surgical use case, in a case where the screen display target is switched between multiple preset gaze targets A1, it is possible, by minimizing the endoscope posture change, to obtain the effects of minimizing the risk of an internal organ interference due to the endoscope moving operation and reducing the risk of inter-instrument interference in the external working space.

<1-6. Action and Effect>

As described above, the medical observation system 1 according to the present embodiment includes: the endoscope 4100 (for example, the imaging unit 12) that acquires the first operative field image (for example, a wide-angle image): the arm unit 11 that supports and moves the endoscope 4100; the gaze target extraction unit 271a that extracts the gaze target A1 from the first operative field image; the gaze point information calculation unit 271b that calculates gaze point information related to the gaze point A3 of the gaze target A1; the movable range determination unit 272a that determines the movable range (endoscope movable range) of the endoscope 4100 enabling cutout of the second operative field image including the gaze point A3 from the first operative field image on the basis of the gaze point information; the camera posture determination unit 272b that determines posture information related to the position and posture of the endoscope 4100 on the basis of the movable range; and the arm control unit 23 that controls the arm unit 11 on the basis of the posture information. This makes it possible to automatically derive the position (for example, the distal end position of the endoscope 4100) and posture of the endoscope 4100 to control the arm unit 11, making it possible to capture the gaze target A1 in the visual field in an appropriate gaze line direction.

Furthermore, the gaze point information calculation unit 271b may calculate the position of the gaze point A3 as gaze point information from the multiple feature points A2 constituting the gaze target A1. This makes it possible to obtain the position of the gaze point A3 with high accuracy and reliability.

In addition, the gaze point information calculation unit 271b may calculate, as the gaze point information, the position of the gaze point A3 and the requested gaze line vector based on the gaze point A3, from the multiple feature points A2 constituting the gaze target A1. This makes it possible to obtain the position of the gaze point A3 with high accuracy and reliability.

Furthermore, the gaze point information calculation unit 271b may calculate the position of the gaze point A3 as the gaze point information on the basis of three-dimensional information of the multiple feature points A2. This makes it possible to obtain the three-dimensional position of the gaze point A3 with high accuracy and reliability.

Furthermore, the gaze point information calculation unit 271b may calculate the three-dimensional information of the multiple feature points A2 on the basis of the position information and the depth information on the image of the multiple feature points A2. This makes it possible to obtain the three-dimensional information of each feature point A2 with high accuracy and reliability.

Furthermore, the gaze point information calculation unit 271b may detect the multiple feature points A2 by instrument recognition processing or organ recognition processing. This makes it possible to automatically detect each feature point A2.

Furthermore, the gaze point information calculation unit 271b may detect multiple feature points A2 in accordance with designation by a user such as a doctor or an assistant. This makes it possible to detect each feature point A2 desired by the user.

Furthermore, in addition to the gaze point information as a basis, the movable range determination unit 272a may determine the movable range on the basis of the position information of the distal end of the endoscope 4100 and the angle information of the cutout maximum oblique-viewing angle of the second operative field image based on the viewing angle of the endoscope 4100. This makes it possible to obtain the movable range with high accuracy and reliability.

Furthermore, the movable range determination unit 272a may set a virtual wall, which is a boundary of a region that restricts changes in the position and posture of the endoscope 4100, on the basis of the boundary of the movable range. With this configuration, even when the distal end or the like of the endoscope 4100 reaches the virtual wall, the movement of the endoscope 4100 beyond the virtual wall can be restricted.

Furthermore, in addition to the gaze point information as a basis, the movable range determination unit 272a may set a virtual wall on the basis of an approach prohibition region that prohibits the endoscope 4100 from approaching the gaze point A3. This makes it possible to prohibit the distal end or the like of the endoscope 4100 from approaching the gaze point A3.

Furthermore, the camera posture determination unit 272b may determine the position and posture of the endoscope 4100 optimizing the tracking of the gaze target A1 and the cutout of the second operative field image on the basis of the gaze point information and the movable range. This makes it possible to appropriate execution of tracking of the gaze target A1 and cutout of the second operative field image. Note that the optimization level of tracking and cutout may be different for each use case or user, for example.

Furthermore, the camera posture determination unit 272b may determine the cutout range of the second operative field image in addition to the position and posture of the endoscope 4100 on the basis of the gaze point information and the movable range, and include the determined cutout range in the posture information. This makes it possible to automatically derive the cutout range, leading to reliable acquisition of the second operative field image.

Moreover, the medical observation system 1 may further include the presentation device 40 that presents the second operative field image. This makes it possible for the user such as a doctor or an assistant to visually recognize the second operative field image.

Furthermore, in a case where the endoscope 4100 exceeds the movable range, the presentation device 40 may output an image (for example, a warning image) indicating that the endoscope 4100 exceeds the movable range. This enables visual recognition of the image indicating that the endoscope 4100 exceeds the movable range, making it possible to grasp that the endoscope 4100 exceeds the movable range.

Furthermore, the gaze target extraction unit 271a may extract multiple gaze targets A1 from the first operative field image, the gaze point information calculation unit 271b may calculate gaze point information related to the gaze point A3 for each gaze target A1, and the movable range determination unit 272a may determine a movable range enabling cutout of the second operative field image for each gaze target A1 from the first operative field image on the basis of the gaze point information. With this configuration, even in the presence of multiple gaze targets A1, the position and posture of the endoscope 4100 can be automatically derived to control the arm unit 11, making it possible to capture the gaze target A1 in the visual field in an appropriate gaze line direction.

Furthermore, the camera posture determination unit 272b may determine the posture information on the basis of the movable range according to the requested level (for example, the ratio value) of the gaze point A3 for each gaze target A1. This makes it possible obtain the posture information with high accuracy and reliability even in the presence of multiple gaze targets A1.

Furthermore, the gaze target extraction unit 271a may extract multiple gaze targets A1 from the first operative field image, the gaze point information calculation unit 271b may calculate gaze point information related to the gaze point A3 for each gaze target A1, and the movable range determination unit 272a may determine a movable range enabling cutout of the second operative field image for each gaze target A1 from the first operative field image on the basis of the gaze point information. With this configuration, even in the presence of multiple gaze targets A1, the position and posture of the endoscope 4100 can be automatically derived to control the arm unit 11, making it possible to capture the gaze target A1 in the visual field in an appropriate gaze line direction.

Furthermore, in a case where the endoscope 4100 moves from the first movable range to the second movable range, among the movable ranges for each gaze target A1, according to the visual field movement from the first gaze point A3 to the second gaze point A3 among the gaze points A3 for each gaze target A1, the arm control unit 23 may control the arm unit 11 to minimize the moving distance of the endoscope 4100. This makes it possible to minimize the position and posture change of the endoscope 4100, leading to achievement of minimization of the risk of internal organ interference due to the moving operation of the endoscope 4100 and reduction of the risk of inter-instrument interference in the external working space.

### 2. OTHER EMBODIMENTS

The processing according to the above-described embodiments (or modifications) may be performed in various different forms (modifications) other than the above-described embodiments. For example, among each process described in the above embodiments, all or a part of the processes described as being performed automatically may be manually performed, or the processes described as being performed manually can be performed automatically by a known method. In addition, the processing procedures, specific names, and information including various data and parameters illustrated in the above Literatures or drawings can be flexibly altered unless otherwise specified. For example, various types of information illustrated in each of the drawings are not limited to the information illustrated.

In addition, each of components of each device is provided as a functional and conceptional illustration and thus does not necessarily need to be physically configured as illustrated. That is, the specific form of distribution/integration of each device is not limited to those illustrated in the drawings, and all or a part thereof may be functionally or physically distributed or integrated into arbitrary units according to various loads and use conditions.

Furthermore, the above-described embodiments (or modifications) can be appropriately combined within a range implementable without contradiction of processes. The effects described in the present specification are merely examples, and thus, there may be other effects, not limited to the exemplified effects.

In the embodiments (or modifications) described above, a system represents a set of multiple constituents (devices, modules (components), or the like), regardless of whether all the constituents are located in a same housing. Therefore, multiple devices housed in separate housings and connected via a network, and one device in which multiple modules are housed in one housing, are both systems.

Furthermore, for example, the embodiments (or modifications) described above can adopt a configuration of cloud computing in which one function is cooperatively shared and processed by multiple devices via a network. Furthermore, individual steps described in the above-described processing (for example, flowcharts) can be executed by one device or can be executed by multiple devices in shared operation.

Furthermore, when one step includes multiple processes, the multiple processes included in the one step can be executed by one device or can be executed by multiple devices in shared operation.

### 3. HARDWARE CONFIGURATION EXAMPLE

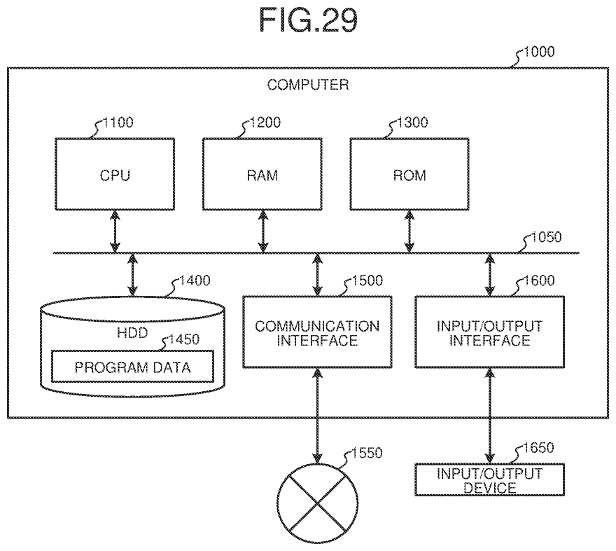

The information processing device such as the control unit 20 described above is actualized by a computer 1000 having a configuration as illustrated in FIG. 29, for example. FIG. 29 is a diagram illustrating a schematic configuration of hardware of the computer 1000. Hereinafter, the control unit 20 according to the embodiment will be described as an example.

As illustrated in FIG. 29, the computer 1000 includes a CPU 1100, RAM 1200, read only memory (ROM) 1300, a hard disk drive (HDD) 1400, a communication interface 1500, and an input/output interface 1600. Individual components of the computer 1000 are interconnected by a bus 1050.

The CPU 1100 operates on the basis of a program stored in the ROM 1300 or the HDD1400 so as to control each of components. For example, the CPU 1100 develops the program stored in the ROM 1300 or the HDD1400 into the RAM 1200 and executes processing corresponding to various programs.

The ROM 1300 stores a boot program such as a basic input output system (BIOS) executed by the CPU 1100 when the computer 1000 starts up, a program dependent on hardware of the computer 1000, or the like.

The HDD1400 is a non-transitory computer-readable recording medium that records a program executed by the CPU 1100, data used by the program, or the like. Specifically, the HDD1400 is a recording medium that records an information processing program according to the present disclosure, which is an example of program data 1450.

The communication interface 1500 is an interface for connecting the computer 1000 to an external network 1550 (for example, the Internet). For example, the CPU 1100 receives data from other devices or transmits data generated by the CPU 1100 to other devices via the communication interface 1500.

The input/output interface 1600 is an interface for connecting between an input/output device 1650 and the computer 1000. For example, the CPU 1100 receives data from an input device such as a keyboard or a mouse via the input/output interface 1600. In addition, the CPU 1100 transmits data to an output device such as a display, a speaker, or a printer via the input/output interface 1600. Furthermore, the input/output interface 1600 may function as a media interface for reading a program or the like recorded on predetermined recording media. Examples of the media include optical recording media such as a digital versatile disc (DVD) or a phase change rewritable disk (PD), a magneto-optical recording medium such as a magneto-optical disk (MO), a tape medium, a magnetic recording medium, and semiconductor memory.

For example, when the computer 1000 functions as the control unit 20 according to the embodiment, the CPU 1100 of the computer 1000 executes the information processing program loaded on the RAM 1200 so as to implement the functions of the control unit 20 or the like. Furthermore, the HDD 1400 stores the information processing program according to the present disclosure or data in the storage unit 14. While the CPU 1100 executes program data 1450 read from the HDD1400, the CPU 1100 may acquire these programs from another device via the external network 1550, as another example.

### 4. SUPPLEMENTARY NOTES

Note that the present technique can also have the following configurations.

(1)

A medical observation system comprising:
an endoscope that acquires a first operative field image;
an arm unit that supports and moves the endoscope;
a gaze target extraction unit that extracts a gaze target from the first operative field image;
a gaze point information calculation unit that calculates gaze point information related to a gaze point of the gaze target;
a movable range determination unit that determines, on the basis of the gaze point information, a movable range of the endoscope enabling cutout of a second operative field image including the gaze point from the first operative field image;
a posture determination unit that determines posture information related to a position and a posture of the endoscope on the basis of the movable range; and
an arm control unit that controls the arm unit on the basis of the posture information.

(2)

The medical observation system according to (1),
wherein the gaze point information calculation unit calculates a position of the gaze point as the gaze point information from multiple feature points constituting the gaze target.

(3)

The medical observation system according to (1),
wherein the gaze point information calculation unit calculates, as the gaze point information, a position of the gaze point and a requested gaze line vector based on the gaze point, from multiple feature points constituting the gaze target.

(4)

The medical observation system according to (2) or (3),
wherein the gaze point information calculation unit calculates the position of the gaze point as the gaze point information on the basis of three-dimensional information of the multiple feature points.

(5)

The medical observation system according to (4),
wherein the gaze point information calculation unit calculates the three-dimensional information of the multiple feature points on the basis of on-image position information and depth information of the multiple feature points.

(6)

The medical observation system according to any one of (2) to (5),
wherein the gaze point information calculation unit detects the multiple feature points by instrument recognition processing or organ recognition processing.

(7)

The medical observation system according to any one of (2) to (5),
wherein the gaze point information calculation unit detects the multiple feature points according to user designation.

(8)

The medical observation system according to any one of (1) to (7),
wherein the movable range determination unit determines the movable range on the basis of position information of a distal end of the endoscope and angle information of a cutout maximum oblique-viewing angle of the second operative field image based on a viewing angle of the endoscope, in addition to the gaze point information.

(9)

The medical observation system according to any one of (1) to (8),
wherein the movable range determination unit sets a virtual wall, which is a boundary of a region restricting a change in a position and a posture of the endoscope, on the basis of a boundary of the movable range.

(10)

The medical observation system according to (9),
wherein the movable range determination unit sets the virtual wall on the basis of an approach prohibition region that prohibits the endoscope from approaching the gaze point, in addition to the gaze point information.

(11)

The medical observation system according to any one of (1) to (10),
wherein the posture determination unit determines a position and a posture of the endoscope optimizing tracking of the gaze target and cutout of the second operative field image on the basis of the gaze point information and the movable range.

(12)

The medical observation system according to any one of (1) to (11),
wherein the posture determination unit determines a cutout range of the second operative field image in addition to the position and posture of the endoscope on the basis of the gaze point information and the movable range, and includes the determined cutout range in the posture information.

(13)

The medical observation system according to any one of (1) to (12), further comprising
a presentation device that presents the second operative field image.

(14)

The medical observation system according to (13),
wherein, in a case where the endoscope exceeds the movable range, the presentation device presents an image indicating that the endoscope exceeds the movable range.

(15)

The medical observation system according to any one of (1) to (14),
wherein the gaze target extraction unit extracts multiple gaze targets from the first operative field image,
the gaze point information calculation unit calculates gaze point information related to the gaze point for each of the gaze targets, and
the movable range determination unit determines, on the basis of the gaze point information, the movable range enabling cutout of the second operative field image for each of the gaze targets from the first operative field image.

(16)

The medical observation system according to (15),
wherein the posture determination unit determines the posture information on the basis of the movable range according to a requested level of the gaze point for each of the gaze targets.

(17)

The medical observation system according to any one of (1) to (14),
wherein the gaze target extraction unit extracts multiple gaze targets from the first operative field image,
the gaze point information calculation unit calculates gaze point information related to the gaze point for each of the gaze targets, and
the movable range determination unit determines, on the basis of the gaze point information, the movable range enabling cutout of the second operative field image from the first operative field image for each of the gaze targets.

(18)

The medical observation system according to (17),
wherein, in a case where the endoscope moves from a first movable range to a second movable range among the movable ranges for each of the gaze targets, according to a visual field movement from a first gaze point to a second gaze point among the gaze points for each of the gaze targets, the arm control unit controls the arm unit to minimize a moving distance of the endoscope.

(19)

An information processing device comprising:
a gaze target extraction unit that extracts a gaze target from a first operative field image obtained by an endoscope;
a gaze point information calculation unit that calculates gaze point information related to a gaze point of the gaze target;
a movable range determination unit that determines, on the basis of the gaze point information, a movable range of the endoscope enabling cutout of a second operative field image including the gaze point from the first operative field image;
a posture determination unit that determines posture information related to a position and a posture of the endoscope on the basis of the movable range; and
an arm control unit that controls an arm unit that supports and moves the endoscope, on the basis of the posture information.

(20)

An information processing method comprising:
extracting a gaze target from a first operative field image obtained by an endoscope;
calculating gaze point information related to a gaze point of the gaze target;
determining, on the basis of the gaze point information, a movable range of the endoscope enabling cutout of a second operative field image including the gaze point from the first operative field image;
determining posture information related to a position and a posture of the endoscope on the basis of the movable range; and
controlling an arm unit that supports and moves the endoscope, on the basis of the posture information.

(21)

A medical observation method using the medical observation system according to any one of (1) to (18).

(22)

An information processing device using the medical observation system according to any one of (1) to (18).

(23)

An information processing method using the medical observation system according to any one of (1) to (18).

### REFERENCE SIGNS LIST

1 MEDICAL OBSERVATION SYSTEM
10 ROBOT ARM DEVICE
11 ARM UNIT
11a JOINT
12 IMAGING UNIT
13 LIGHT SOURCE UNIT
14 STORAGE UNIT
20 CONTROL UNIT
21 IMAGE PROCESSING UNIT
22 IMAGING CONTROL UNIT
23 ARM CONTROL UNIT
24 DISPLAY CONTROL UNIT
25 RECEPTION UNIT
26 DISPLAY CONTROL UNIT
27 GAZE PROCESSING UNIT
40 PRESENTATION DEVICE
60 STORAGE UNIT
271 GAZE INFORMATION PROCESSING UNIT
271a GAZE TARGET EXTRACTION UNIT
271b GAZE POINT INFORMATION CALCULATION UNIT
272 MOTION LINKING CONTROL UNIT
272a MOVABLE RANGE DETERMINATION UNIT
272b CAMERA POSTURE DETERMINATION UNIT
4100 ENDOSCOPE
5000 ENDOSCOPIC SURGERY SYSTEM
5001 ENDOSCOPE
5003 LENS BARREL
5005 CAMERA HEAD
5007 LENS UNIT
5009 IMAGING UNIT
501₁ DRIVE UNIT
5013 COMMUNICATION UNIT
5015 CAMERA HEAD CONTROL UNIT
5017 SURGICAL TOOL
5019 INSUFFLATION TUBE
502₁ ENERGY TREATMENT TOOL
5023 FORCEPS
5025a TROCAR
5025b TROCAR
5025c TROCAR
5025d TROCAR
5027 SUPPORT ARM DEVICE
5029 BASE UNIT
503₁ ARM UNIT
5033a JOINT
5033b JOINT
5033c JOINT
5035a LINK
5035b LINK
5037 CART
504₁ DISPLAY DEVICE
5043 LIGHT SOURCE DEVICE
5045 ARM CONTROL DEVICE
5047 INPUT DEVICE
5049 TREATMENT TOOL CONTROL DEVICE
5051 INSUFFLATOR
5053 RECORDER
5055 PRINTER
5057 FOOT SWITCH
5059 COMMUNICATION UNIT
5061 IMAGE PROCESSING UNIT
5063 CONTROL UNIT
5065 TRANSMISSION CABLE
5067 SURGEON
5069 PATIENT BED
5071 PATIENT
A1 GAZE TARGET
A2 FEATURE POINT
A3 GAZE POINT
A4 REQUESTED GAZE LINE VECTOR
B1 OBSTACLE
C1 ENDOSCOPE INSERTION POINT
C2 ENDOSCOPE DISTAL END POINT
C3 VIEWING ANGLE
C4 MAXIMUM OBLIQUE-VIEWING ANGLE
C5 INSCRIBE ANGLE
D1 STRAIGHT LINE
D2 CUTOUT GAZE LINE VECTOR
G1 IMAGE
G2 IMAGE
G3 IMAGE
R1 WIDE-ANGLE VISUAL FIELD
R2 DISPLAY TARGET REGION

1. A medical observation system comprising:
an endoscope that acquires a first operative field image;
an arm unit that supports and moves the endoscope;
a gaze target extraction unit that extracts a gaze target from the first operative field image;
a gaze point information calculation unit that calculates gaze point information related to a gaze point of the gaze target;
a movable range determination unit that determines, on the basis of the gaze point information, a movable range of the endoscope enabling cutout of a second operative field image including the gaze point from the first operative field image;
a posture determination unit that determines posture information related to a position and a posture of the endoscope on the basis of the movable range; and
an arm control unit that controls the arm unit on the basis of the posture information.

2. The medical observation system according to claim 1,
wherein the gaze point information calculation unit calculates a position of the gaze point as the gaze point information from multiple feature points constituting the gaze target.

3. The medical observation system according to claim 1,
wherein the gaze point information calculation unit calculates, as the gaze point information, a position of the gaze point and a requested gaze line vector based on the gaze point, from multiple feature points constituting the gaze target.

4. The medical observation system according to claim 2,
wherein the gaze point information calculation unit calculates the position of the gaze point as the gaze point information on the basis of three-dimensional information of the multiple feature points.

5. The medical observation system according to claim 4,
wherein the gaze point information calculation unit calculates the three-dimensional information of the multiple feature points on the basis of on-image position information and depth information of the multiple feature points.

6. The medical observation system according to claim 2,
wherein the gaze point information calculation unit detects the multiple feature points by instrument recognition processing or organ recognition processing.

7. The medical observation system according to claim 2,
wherein the gaze point information calculation unit detects the multiple feature points according to user designation.

8. The medical observation system according to claim 1,
wherein the movable range determination unit determines the movable range on the basis of position information of a distal end of the endoscope and angle information of a cutout maximum oblique-viewing angle of the second operative field image based on a viewing angle of the endoscope, in addition to the gaze point information.

9. The medical observation system according to claim 1,
wherein the movable range determination unit sets a virtual wall, which is a boundary of a region restricting a change in a position and a posture of the endoscope, on the basis of a boundary of the movable range.

10. The medical observation system according to claim 9,
wherein the movable range determination unit sets the virtual wall on the basis of an approach prohibition region that prohibits the endoscope from approaching the gaze point, in addition to the gaze point information.

11. The medical observation system according to claim 1,
wherein the posture determination unit determines a position and a posture of the endoscope optimizing tracking of the gaze target and cutout of the second operative field image on the basis of the gaze point information and the movable range.

12. The medical observation system according to claim 1,
wherein the posture determination unit determines a cutout range of the second operative field image in addition to the position and posture of the endoscope on the basis of the gaze point information and the movable range, and includes the determined cutout range in the posture information.

13. The medical observation system according to claim 1, further comprising
a presentation device that presents the second operative field image.

14. The medical observation system according to claim 13,
wherein, in a case where the endoscope exceeds the movable range, the presentation device presents an image indicating that the endoscope exceeds the movable range.

15. The medical observation system according to claim 1,
wherein the gaze target extraction unit extracts multiple gaze targets from the first operative field image,
the gaze point information calculation unit calculates gaze point information related to the gaze point for each of the gaze targets, and
the movable range determination unit determines, on the basis of the gaze point information, the movable range enabling cutout of the second operative field image for each of the gaze targets from the first operative field image.

16. The medical observation system according to claim 15,
wherein the posture determination unit determines the posture information on the basis of the movable range according to a requested level of the gaze point for each of the gaze targets.

17. The medical observation system according to claim 1,
wherein the gaze target extraction unit extracts multiple gaze targets from the first operative field image,
the gaze point information calculation unit calculates gaze point information related to the gaze point for each of the gaze targets, and
the movable range determination unit determines, on the basis of the gaze point information, the movable range enabling cutout of the second operative field image from the first operative field image for each of the gaze targets.

18. The medical observation system according to claim 17,
wherein, in a case where the endoscope moves from a first movable range to a second movable range among the movable ranges for each of the gaze targets, according to a visual field movement from a first gaze point to a second gaze point among the gaze points for each of the gaze targets, the arm control unit controls the arm unit to minimize a moving distance of the endoscope.

19. An information processing device comprising:
a gaze target extraction unit that extracts a gaze target from a first operative field image obtained by an endoscope;
a gaze point information calculation unit that calculates gaze point information related to a gaze point of the gaze target;
a movable range determination unit that determines, on the basis of the gaze point information, a movable range of the endoscope enabling cutout of a second operative field image including the gaze point from the first operative field image;
a posture determination unit that determines posture information related to a position and a posture of the endoscope on the basis of the movable range; and
an arm control unit that controls an arm unit that supports and moves the endoscope, on the basis of the posture information.

20. An information processing method comprising:
extracting a gaze target from a first operative field image obtained by an endoscope;
calculating gaze point information related to a gaze point of the gaze target;
determining, on the basis of the gaze point information, a movable range of the endoscope enabling cutout of a second operative field image including the gaze point from the first operative field image;
determining posture information related to a position and a posture of the endoscope on the basis of the movable range; and
controlling an arm unit that supports and moves the endoscope, on the basis of the posture information.
